(19) United States
(12) Patent Application Publication
(10) Pub. No.: US 2026/0263061 A1
(43) Pub. Date: Sep. 10, 2026

(54) ADJUSTABLE SUTURE NET FOR SUBSCAPULARIS REPAIR

(71) Applicant: Responsive Arthroscopy, Inc., Minneapolis, MN (US)

(72) Inventors: Aaron Pidde, Bloomington, MN (US); Kimberly Bahoora, Medina, MN (US); Jacob Hustedt, Sandy, UT (US); Douglas Kohrs, Minneapolis, MN (US)

(21) Appl. No.: 19/549,922

(22) Filed: Feb. 25, 2026

Related U.S. Application Data
(63) Continuation of application No. PCT/US2024/044875, filed on Aug. 30, 2024.
(60) Provisional application No. 63/580,263, filed on Sep. 1, 2023.

(51) Int. Cl. A61B 17/04

(52) U.S. Cl. CPC A61B 17/0401; A61B 2017/0464

(57) ABSTRACT

Improved suture anchor systems and methods of use are disclosed herein. The suture anchor system includes a suture containing one or more opened sections each including an opening. A suture net can be formed by passing another section of a same suture or by passing a different suture through each opening included in the one or more opened sections, so that legs of the one or more opened sections interweave with the other section of the same suture or with the different suture to form a suture net. The formed suture net can be further tightened through tensioning ends of the suture(s) through a set of anchors, which can be used to secure a soft tissue to a bone.

### CROSS-REFERENCE TO RELATED APPLICATIONS

This application is a continuation of International Application No. PCT/US2024/044875, filed on Aug. 30, 2024, which claims priority to, and the benefit of, U.S. Provisional Patent Application No. 63/580,263, filed on Sep. 1, 2023, the entireties of all of which are incorporated herein by reference.

### BACKGROUND

The subscapularis is the most powerful muscle in the rotator cuff and is responsible for helping to internally rotate the arm. The rotator cuff is a The subscapularis is the most powerful muscle in the rotator cuff and is responsible for helping to internally rotate the arm. The rotator cuff is a confluence of tendons that connect the muscles originating around the scapula and inserting on the upper humerus. When activated, these muscles raise, lower, and rotate the arm. The rotator cuff tendons together form a cuff that encapsulates the article surface at the top of the humerus. The acromion forms a bony and ligamentous arch over the rotator cuff and is bordered by the acromioclavicular ligament, the coracoid, and the acromioclavicular joint. If the subscapularis muscle is torn or resected from the rotator cuff, subscapularis repair is vitally important to restore the anatomy of the shoulder and to achieve the best functional outcome possible.

Subscapularis repair however can be difficult to perform, due to the small space in which it is contained. It is therefore desirable to design a subscapularis repair system that consumes less space.

### SUMMARY

Provided herein are improved suture anchors, systems, and associated methods for using the improved suture anchors in attaching soft tissue to associated bone or bones. While advent of arthroscopy has allowed for significant advancements in the diagnosis and treatment of subscapularis tears, all-arthroscopic repair of the subscapularis tendon remains a technically demanding procedure. Double-row repair offers the most secure fixation and, therefore, provides an environment that maximizes the potential for improved functional outcomes and decreased rates of rerupture. Arthroscopic and/or other (e.g., open procedures) double-row rotator cuff repair may utilize two rows of anchors, one being medial and the other being lateral, in order to provide better anatomical footprint restoration. For example, in an exemplary open or arthroscopic existing subscapularis repair system, surgeons sometimes use three medial row all-suture anchors and two lateral row polyether ether ketone (PEEK) anchors to provide necessary suture surface area and compression. These subscapularis repair systems, however, generally require a relatively large space in a small shoulder area, which makes the actual subscapularis repair processes challenging. In addition, the number of anchors required in such subscapularis repair systems is also relatively large (e.g., three or more medial row anchors), which may be quite challenging if a patient has a small bone due to age or other possible reasons. Moreover, additional medial row anchors may require more apertures to be formed through the subscapularis tendon (as described herein), thereby making the tendon more susceptible to failure. The relatively large number of anchors may also place undue stress on the humeral bone or increase the likelihood of contacting a total shoulder implant.

It is appreciated herein that it is desirable to design a suture anchor system that will provide sufficient suture surface area coverage and/or compressive force, while consuming less overall space and/or require fewer anchors for application in a small area for repair processes. Embodiments of improved suture anchor systems discussed herein have been designed to limit the number of anchors in use and/or with less space required in subscapularis repair processes by including one or more suture nets in the processes. In some embodiments, an anchor system includes a suture containing one or more opened sections each including an opening. A suture net is then formed by passing another section of the same suture or by passing a different suture through at least one opening included in the one or more opened sections, so that legs (that define each opened section and the opening therebetween, as described herein) of the one or more opened sections interweave with the other section of the same suture or with the different suture to form a suture net. The formed suture net may be further tightened through tensioning ends of the suture(s) through a set of anchors, which can be used to secure a soft tissue to a bone. The number of the required anchors may be decreased without sacrificing the necessary suture surface area and compression due to the formed suture net, which may actually increase the suture surface area within a small area, and may also provide additional compressive force on the soft tissue against the respective bone (e.g., subscapularis tendon on the humerus bone).

Provided herein is a suture anchor system comprising a suture having a first end, a second end, and a first opened section therebetween, the first opened section defining a first leg, a second leg, and a first opening therebetween; and a plurality of anchors, each configured to be independently inserted within a respective tissue (e.g. bone) of a subject about a repair region thereof, the plurality of anchors comprising a first, second, and third anchor, where the first anchor and the second anchor are configured to secure a primary segment of the suture to the respective tissue (e.g. bone), the primary segment including the first end and the first leg of the first opened section; and the second anchor and the third anchor are configured to secure a secondary segment of the suture, the secondary segment extending from the second anchor, passing through the first opening, and through the third anchor to the second end to form a first suture net, such that tensioning the second end when passed through the third anchor transitions the first opened section from a relaxed configuration to a tensioned configuration, thereby allowing a first compressive force to be applied about the repair region by the formed first suture net including the primary segment, the secondary segment, and the second leg of the first opened section.

In some embodiments, tensioning the second end causes the secondary segment to interact with the second leg of the first opened section, thereby enlarging the first opening defined within the first opened section. In some embodiments, the first compressive force applied is adjustable based on the tensioning of the second end, a first distance between the first anchor and the second anchor, a second distance between the second anchor and the third anchor, or any combination thereof.

In some embodiments, one or more of the first, second, and third anchors are secured to the respective bone using an all-suture assembly. In some embodiments, one or more of the first, second, and third anchors are secured to the respective bone using a knotless suture assembly. In some embodiments, the first anchor and the third anchor are disposed on a lateral row of a humerus, and the second anchor is disposed on a medial row of the humerus.

In some embodiments, the suture comprises a second opened section configured to receive the secondary segment therethrough. In some embodiments, the second opened section comprises (i) a third leg disposed along the primary segment, (ii) a fourth leg, and (iii) a second opening therebetween, and where the secondary segment extends from the second anchor, passes through the first opening, second opening, and through the third anchor to the second end, such that tensioning the second end when passed through the third anchor allows for the first compressive force to be applied about the repair region by the primary segment, the secondary segment, the second leg, and the fourth leg.

In some embodiments, the suture comprises three or more opened sections. In some embodiments, at least one opened section is a bifurcation of the suture. In some embodiments, at least one opened section comprises a first separate suture strand affixed to the suture at a proximal and distal location, thereby forming a respective opening between the suture and the first separate strand, the first separate strand, therefore, acting as a respective leg for said at least one opened section. In some embodiments, the first separate strand is affixed to the suture via tying, braiding, an adhesive, or any combination thereof. In some embodiments, the second leg is longer than the first leg.

In some embodiments, the suture anchor system further comprises a second suture having a third end, a fourth end, and a third opened section therebetween, the third opened section defining a fifth leg, a sixth leg, and a third opening therebetween, where the third anchor and the second anchor are configured to secure a primary segment of the second suture to the respective bone, the primary segment of the second suture including the third end and the fifth leg of the third opened section; and the second anchor and the first anchor are configured to secure a secondary segment of the second suture, the secondary segment of the second suture extending from the second anchor, through the third opened section, and through the first anchor to the fourth end to form a second suture net, such that tensioning the fourth end when passed through the first anchor transitions the third opened section from a relaxed configuration to a tensioned configuration, thereby allowing a second compressive force to be applied about the repair region by the formed second suture net including the primary segment of the second suture, the secondary segment of the second suture, and the sixth leg of the third opened section.

In some embodiments, the secondary segment of the second suture is configured to pass through one or both of the first and second opened sections. In some embodiments, tensioning the fourth end causes the secondary segment of the second suture to interact with the sixth leg, thereby enlarging the third opening defined within the third opened section. In some embodiments, the second compressive force applied is adjustable based on the tensioning of the fourth end, a first distance between the first anchor and the second anchor, a second distance between the second anchor and the third anchor, or any combination thereof.

In some embodiments, one or more of the third, second, and first anchors are secured to the respective bone using an all-suture assembly. In some embodiments, one or more of the first, second, and third anchors are secured to the respective bone using a knotless suture assembly. In some embodiments, the first anchor and the third anchor are disposed on a lateral row of a humerus, and the second anchor is disposed on a medial row of the humerus.

In some embodiments, the second suture comprises a fourth opened section configured to receive the secondary segment of the second suture therethrough. In some embodiments, the fourth opened section comprises (i) a seventh leg disposed along the primary segment of the second suture, and (ii) an eighth leg, and wherein the secondary segment of the second suture extending from the second anchor, through the third opened section, the fourth opened section, and through the first anchor to the fourth end such that tensioning the fourth end when passed through the first anchor allows for the second compressive force to be applied about the repair region by the primary segment of the second suture, the secondary segment of the second suture, the sixth leg, and the eighth leg.

In some embodiments, the second suture comprises three or more opened sections. In some embodiments, at least one opened section is a bifurcation of the second suture.

In some embodiments, wherein at least one opened section comprises a second separate suture strand affixed to the second suture at a proximal and distal location thereof, thereby forming a respective opening between the second suture and the second separate strand, the second separate strand therefore acting as a respective leg for said at least one opened section. In some embodiments, the second separate suture strand is affixed to the second suture via tying, braiding, an adhesive, or any combination thereof.

In some embodiments, the suture anchor system further comprising a second suture having a third end, a fourth end, and a third opened section therebetween, the third opened section defining a fifth leg, a sixth leg, and a third opening therebetween, where the third anchor and a fourth anchor are configured to secure a primary segment of the second suture to the respective bone, the primary segment of the second suture including the third end and the fifth leg of the third opened section; and the fourth anchor and the first anchor are configured to secure a secondary segment of the second suture, the secondary segment of the second suture extending from the fourth anchor, through the third opened section, and through the first anchor to the fourth end to form a third suture net, such that tensioning the fourth end when passed through the first anchor transitions the third opened section from a relaxed configuration to a tensioned configuration, thereby allowing a second compressive force to be applied about the repair region by the formed third suture net including the primary segment of the second suture, the secondary segment of the second suture, and the sixth leg of the third opened section.

In some embodiments, tensioning the fourth end causes the secondary segment of the second suture to interact with the sixth leg, thereby enlarging the third opening defined within the third opened section. In some embodiments, the second compressive force applied is adjustable based on the tensioning of the fourth end, a first distance between the first anchor and the fourth anchor, a second distance between the fourth anchor and the third anchor, or any combination thereof.

In some embodiments, one or more of the third, fourth and first anchors are secured to the respective bone using an all-suture assembly. In some embodiments, one or more of the first, fourth, and third anchors are secured to the respective bone using a knotless suture assembly. In some embodiments, the first anchor and the third anchor are disposed on a lateral row of a humerus, and the fourth anchor is disposed on a medial row of the humerus.

In some embodiments, the second suture comprises a fourth opened section configured to receive the secondary segment of the second suture therethrough. In some embodiments, the fourth opened section comprises (i) a seventh leg disposed along the primary segment of the second suture, and (ii) an eighth leg, such that tensioning the fourth end when passed through the first anchor allows for the second compressive force to be applied about the repair region by the primary segment of the second suture, the secondary segment of the second suture, the sixth leg, and the eighth leg.

In some embodiments, the second suture comprises three or more opened sections. In some embodiments, at least one opened section is a bifurcation of the second suture. In some embodiments, at least one opened section comprises a second separate suture strand affixed to the second suture at a proximal and distal location thereof, thereby forming a respective opening between the second suture and the second separate strand, the second separate strand, therefore, acting as a respective leg for said at least one opened section. In some embodiments, the second separate suture strand is affixed to the second suture via tying, braiding, an adhesive, or any combination thereof

Provided herein, in some embodiments, is a suture anchor system comprising a first suture having a first end, a second end, and a plurality of opened sections therebetween, each opened section having a respective first leg, a respective second leg, and a respective opening therebetween; a plurality of anchors, each configured to be independently inserted within a respective bone of a subject about a repair region thereof, the plurality of anchors comprising a first, second, and third anchor; and a second suture comprising a third and fourth end, the second suture configured to be secured to the respective bone via the first, second, and third anchors, where the first anchor and the second anchor are configured to secure a primary segment of the first suture to the respective bone, the primary segment including the first end and a first leg of one or more opened sections of the plurality of opened sections; the second anchor and the third anchor are configured to secure a secondary segment of the first suture, the secondary segment including second end and a first leg of one or more opened sections of the plurality of opened sections that are different from the one or more opened sections of the primary segment; and the second suture is configured to be passed through the first anchor, the second anchor, the third anchor, and one or more respective openings of each of the plurality of opened sections on the primary and secondary segments to form a suture net, such that tensioning the first suture, the second suture, or both, when passed through the first anchor, the third anchor, or both, transitions one or more opened sections on the primary and/or secondary segments from a relaxed configuration to a tensioned configuration, thereby allowing a compressive force to be applied about the repair region by the formed suture net including the primary segment, the secondary segment, and the second leg of the one or more of the plurality of opened sections.

In some embodiments, tensioning the second suture causes the second suture to interact with the second leg of the one or more opened sections, thereby enlarging the respective opening defined within the one or more opened sections. In some embodiments, the compressive force applied is adjustable based on the tensioning of the second suture, a first distance between the first anchor and the second anchor, a second distance between the second anchor and the third anchor, or any combination thereof.

In some embodiments, one or more of the first, second, and third anchors are secured to the respective bone using an all-suture assembly. In some embodiments, one or more of the first, second, and third anchors are secured to the respective bone using a knotless suture assembly. In some embodiments, the first anchor and the third anchor are disposed on a lateral row of a humerus, and the second anchor is disposed on a medial row of the humerus.

In some embodiments, at least one opened section is a bifurcation of the suture. In some embodiments, at least one opened section comprises a third suture affixed to the suture at a proximal and distal location, thereby forming a respective opening between the first suture and the third suture, the third suture therefore acting as a respective leg for said at least one opened section. In some embodiments, the third suture is affixed to the first suture via tying, braiding, an adhesive, or any combination thereof.

In some embodiments, the second leg has a same length as the first leg for each of the one or more opened sections. In some embodiments, the second leg is longer than the first leg among one or more of the one or more opened sections.

Provided herein are methods of anchoring a soft tissue to a bone. An exemplary method comprises inserting a second anchor within a bone of a subject; passing a suture through the second anchor, the suture having a first end and second end extending from the second anchor; passing the first and second ends of the suture through a soft tissue to be secured to the bone, such that the suture passes from a bone facing portion of the soft tissue and through an aperture to an opposite facing portion of the soft tissue; securing the first end of the suture through a first anchor that is then inserted into the bone, wherein the suture from the first end to the aperture comprises an opened section having a first leg, a second leg, and an opening therebetween, the suture defining a primary segment from the first end to the aperture and comprising the first leg; passing at least a portion of a secondary segment of the suture through the opening of the opened section and then through a third anchor that is then inserted into the bone to form a suture net, the secondary segment comprising the suture from the second end to the aperture; tensioning the secondary segment so as to transition the opened section from a relaxed configuration to a tensioned configuration via interaction between the secondary segment and the second leg, such that a compressive force is applied about the tissue on the bone by formed suture net including the primary segment, the secondary segment, and the second leg; and securing the second end to the bone via one or more of the first, second, and third anchors.

In some embodiments, the method further comprises forming one or more additional suture nets using any combination of the first, the second, the third, and/or one or more additional anchors. In some embodiments, the formed suture net and/or one or more additional suture nets comprise any combination of features of the suture anchor system described above.

Provided herein are alternative methods of anchoring a soft tissue to a bone. An exemplary method comprises inserting a second anchor within a bon e of a subject; passing a first suture and second suture through the second anchor, the first suture having a first and second end extending from the second anchor, the second suture having a third and fourth end extending from the second anchor; passing the first, second, third, and fourth ends through a soft tissue to be secured to the bone, such that the first suture and the second suture passes from a bone facing portion of the soft tissue and through an aperture to an opposite facing portion; passing the first end of the first suture through a first anchor that is then inserted into the bone, wherein the first suture from the first end to the aperture comprises a first opened section having one or more openings therebetween, the first suture defining a primary segment from the first end to the aperture; passing the second end of the first suture through a third anchor that is then inserted into the bone, wherein the first suture from the second end to the aperture comprises a second opened section having one or more openings, the first suture defining a secondary segment from the second end to the aperture; passing the third end of the second suture through one or more of the one or more openings of the first opened section and the one or more openings of the second opened section, and then through one of the first anchor and the third anchor; passing the fourth end of the second suture through remaining one or more of the one or more openings of the first opened section and the one or more openings of the second opened section, and then through the other of the first anchor and the third anchor to form a suture net; tensioning the first suture and the second suture so as to transition the second suture and the first and second opened sections from a relaxed configuration to a tensioned configuration via interaction between the second suture and the one or more openings in the first opened sections and the one or more openings in the second opened section, such that a compressive force is applied about the tissue on the bone by the formed suture net including the first suture and the second suture; and securing the first, second, third, and four ends of the first and second sutures to the bone via the first anchor and the third anchor.

In some embodiments, the method further comprises forming one or more additional suture nets using any combination of the first, the second, the third, and/or one or more additional anchors. In some embodiments, the formed suture net and/or one or more additional suture nets comprise any combination of features of the suture anchor system described above.

These and other embodiments are described in further detail in the following description related to the appended drawing figures.

### BRIEF DESCRIPTION OF THE DRAWINGS

Specific embodiments of the disclosed devices, delivery systems, or methods will now be described with reference to the drawings. The patent or application file contains at least one drawing executed in color. Copies of this patent or patent application publication with color drawing(s) will be provided by the Office upon request and payment of the necessary fee. Nothing in this detailed description is intended to imply that any particular component, feature, or step is essential to the invention.

FIG. 1A shows a schematic view of a suture anchor system (e.g., repair system) containing a suture net and a separate suture, in accordance with some embodiments.

FIG. 1B shows a schematic view of the suture anchor system from FIG. 1A placed over a subscapularis tendon repair site.

FIG. 2A shows an exemplary suture having an opened section for forming a suture net, in accordance with some embodiments.

FIGS. 2B-2D show a process for forming a suture net using the suture shown in FIG. 2A, in accordance with some embodiments.

FIG. 3A shows a schematic view of a suture net, in accordance with some embodiments.

FIG. 3B shows an exemplary application of a suture net about a subscapularis tendon, in accordance with some embodiments.

FIGS. 4A-4C show another exemplary process for forming a suture net, in accordance with some embodiments.

FIGS. 5A-5C show exemplary opened sections with one or more associated loops for forming a suture net, in accordance with some embodiments.

FIG. 6 shows a flow chart of an exemplary method for forming a suture net about a soft tissue repair site, in accordance with some embodiments.

FIG. 7 shows a flow chart of another exemplary method for forming a suture net about a soft tissue site, in accordance with some embodiments.

FIG. 8 shows an exemplary pre-formed suture net, in accordance with some embodiments.

FIGS. 9A-B show an exemplary system of securing two or more bifurcated sutures.

FIG. 10 shows an exemplary embodiment of a system for securing two or more sutures with bifurcated nets.

FIGS. 11A-B show top and bottom views of the system shown in FIG. 10.

FIG. 12 illustrates a flowchart of a method of securing two or more bifurcated sutures.

### DETAILED DESCRIPTION

Specific embodiments of the disclosed device and method of use will now be described with reference to the drawings. Nothing in this detailed description is intended to imply that any particular component, feature, or step is essential to the disclosure.

Embodiments of the present method and apparatus can be used to repair and reconstruct torn ligaments and tendons in a variety of locations of a human body. The subscapularis tendon is selected in the following description of subscapularis repair systems because of the complexity of the human shoulder. It will be appreciated that the methods and apparatus disclosed herein may have many other possible applications, including other anatomical parts of a person, such as for example, the achilles tendon, used with bronstram repair, etc.

Subscapularis muscle is one of four rotator cuff muscles, and is located at the front of a human shoulder. One of the primary roles of the subscapularis muscle is to turn an arm inwards and is a key muscle in doing any lifting movements across a human's chest or twisting inwards. The subscapularis muscle is essential for overhead sports, such as swimming, racquet sports and throwing. Tears of the subscapularis tendon are less common than supraspinatus and infraspinatus tendon tears (commonly known as “rotator cuff tears”). However, tears of the subscapularis tendon can be much more painful, since the restraints to the long head of biceps tendon are often also torn and the biceps tendon dislocates from its groove. The biceps tendon then also becomes painful and weak. In some cases, the subscapularis tendon is purposely torn so as to perform a surgery about the shoulder region (e.g., shoulder replacement, etc.).

Subscapularis tear is generally repaired by suturing it back to the bone (e.g., humerus bone). As the largest & strongest rotator cuff muscle, the subscapularis muscle can provide about 53% of the total rotator cuff strength. Accordingly, for subscapularis repair, it is desirable that the applied repair systems can provide a good suture surface area and compression.

The present disclosure, in some embodiments, provides subscapularis repair systems and methods that consume less overall space without sacrificing the required suture surface area and compression imposed on the subscapularis tendon. In some embodiments, the subscapularis repair system disclosed herein requires only one or two medial row anchors for anchoring the sutures to achieve good suture surface area and compression (provided on the subscapularis tendon onto the humerus bone) in subscapularis repair (as opposed to three, four, or more medial row anchors that consume more space). In some embodiments, a disclosed subscapularis repair system incorporates a “suture net” structure, so as to increase surface area coverage and/or compression applied across the subscapularis repair site. In some embodiments, application of the suture net includes a suture passing through one or more loops or openings formed between the medial row anchor(s) and lateral row anchor(s) before being fixed or secured by these anchors.

Provided herein are improved suture anchors, systems, and associated methods for using the improved suture anchors in attaching soft tissue to associated bone or bones. As used herein, soft tissue refers to, at least, soft tissues that connect, support, and surround the different body organs, which may include fat, muscle, nerves, blood vessels, ligaments, tendons, and/or other fibrous soft tissues. Soft tissue is distinguishable herein from bone, or hard tissue. The term “tissue” when used alone, may refer to soft or hard tissue, depending on the circumstance, as would be obvious to one of skill in the art upon reading the disclosures herein.

The word “leg” as used throughout this disclosure refers to an extending portion of a suture with respect to a suture origin point and is interchangeably used with the word “limb” herein.

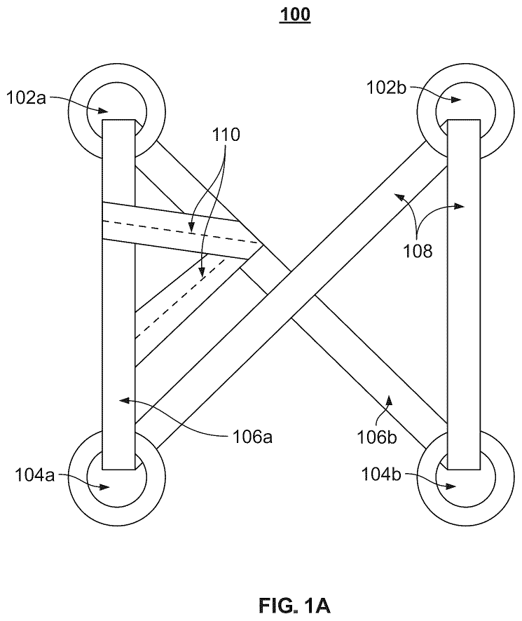

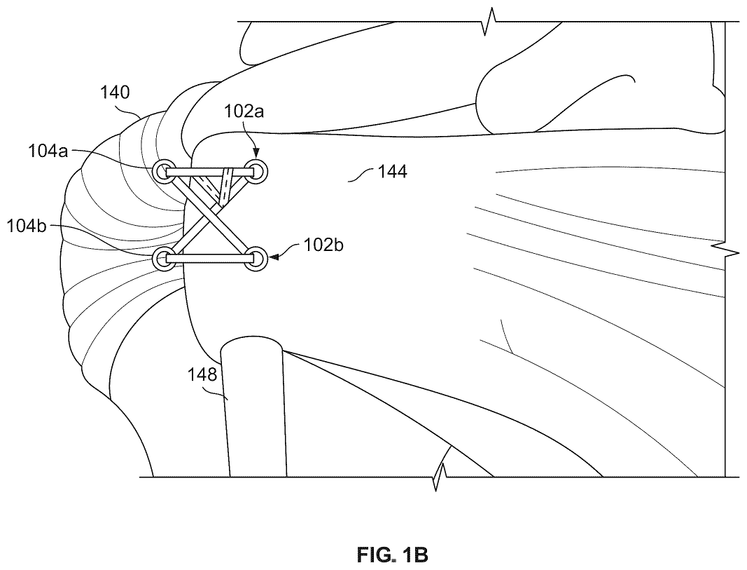

FIG. 1A illustrates an exemplary subscapularis repair system 100 with a suture net, according to some embodiments described herein. In some embodiments, the repair system 100 includes two medial row anchors 102a and 102b (together or individually also referred to as “medial row anchor 102”) and two lateral row anchors 104a and 104b (together or individually also referred to as “lateral row anchor 104”). In some embodiments, any number of medial row anchors and lateral row anchors can be used. For example, in some embodiments, a subscapularis repair system described herein includes one or more medial row anchors with one or more lateral row anchors, such as one medial row anchor and one, two, three, four, five, or more lateral row anchors, or one lateral row anchor, and one, two, three, four, five, or more medial row anchors.

With reference to repair system 100, the two medial row anchors 102a and 102b each hold at least a portion of a respective suture or a respective suture net. For example, the medial row anchor 102b in FIG. 1A holds a portion of suture 108, while the medial row anchor 102a in the figure holds a portion of a suture net formed by a suture 106 and a net structure 110 (together referred to as a suture net). In some embodiments, a suture 106,108 held by a medial row anchor 102a-b can freely slide within the anchor.

In some embodiments, for any repair system described herein, a suture may include one or more opened sections across a length of said suture (e.g., see suture 106a with opened section defined by 110). In some embodiments, an end of said suture is configured to pass through the one or more opened sections so as to form the suture net, as described herein (see e.g., 106b). For example, in FIG. 1, for the left medial row anchor 102a that holds a suture net, a certain portion of the suture (e.g., 106a) may include an opened section.

FIG. 1B depicts the subscapularis repair system as disposed about a repair site, wherein the two medial anchors 102a-b are secured to the humerus bone 140, and wherein the sutures 106a-b, 108 passes through the subscapularis tendon 144 and are secured at another location on the humerus bone 140 using lateral anchors 104a-b. Also shown is bicep muscle 148 for reference.

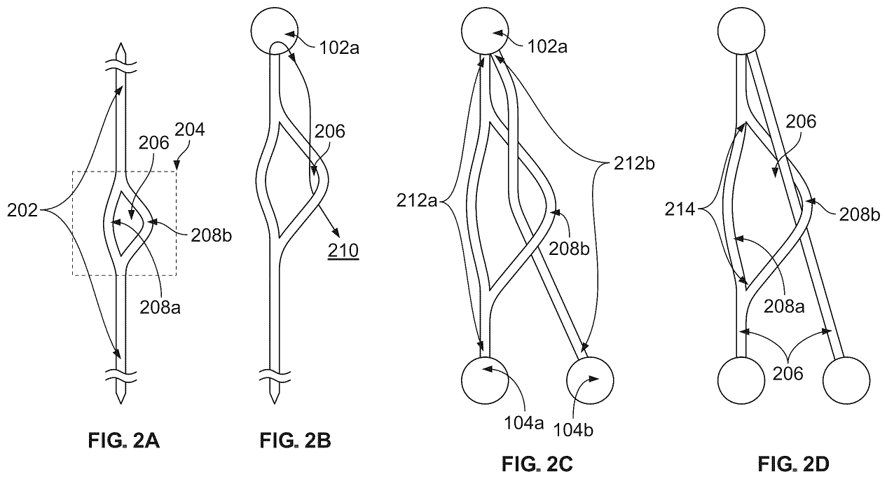

As shown in FIG. 2A, a suture 202, which may be used for any repair system described herein, may include an opened section 204 that forms a loop or opening 206 along the suture 202. In some embodiments, the opened section 204 includes two legs (also referred to as “strands”) 208a and 208b that define the opening 206 therebetween. In some embodiments, the leg lengths of the two legs 208a and 208b forming the opening 206 have equal lengths. In some embodiments, the leg lengths of the two legs 208a and 208b have unequal leg lengths. For example, in the embodiment illustrated in FIG. 2A, a second leg 208b (or the right leg) of the opened section 204 has a larger length when compared to a first leg 208a (or the left leg) of the opened section. In some embodiments, the suture 202 corresponds to the suture 106 as depicted in FIG. 1.

FIGS. 2B-2D further illustrate a process for forming a suture net using the suture 202 illustrated in FIG. 2A, according to some embodiments of a repair system described herein. Specifically, FIG. 2B illustrates a front end of the bifurcated suture 202 extending from a medial row anchor 102a (e.g., as depicted in FIG. 2B) and then through the opening 206 formed by the opened section (as indicated by arrow 210 in FIG. 2B). In some embodiments, the front end of the suture 202 may be also referred to as the second end of the suture, where the first end of the suture 202 may be secured via a first lateral row anchor (e.g., first lateral row anchor 104a shown in FIG. 2C). As depicted in FIG. 2B, the opened section including the opening 206 formed therein does not pass through the medial row anchor 102a. In some embodiments, with reference to FIG. 2C, a segment of the suture 202 between the medial row anchor 102a and the first lateral row anchor 104a may be referred to as a primary segment or first segment 212a, while a segment of the suture 202 between the medial row anchor 102a may be referred to as a secondary segment or second segment 212b.

FIG. 2C further illustrates the secondary segment 212b of the suture 202 having passed through and secured via the second lateral row anchor 104b, which is different from the first lateral row anchor 104a that secures or secured the primary segment of the suture 202. As seen in FIG. 2C, after the secondary segment of the suture 202 passes through the opening 206 and is passed through the second lateral row anchor 104b, a preliminary, un-tensioned configuration of a net structure is formed, which includes interweaves formed by the opened section 204 (e.g., the second leg or right leg 208b) of the suture 202 and the secondary segment 212b of the suture. In some embodiments, the as-formed net structure provides an increased suture surface area coverage about the repair site (e.g., subscapularis tendon situated over the humerus bone) due to the formed interweaves (e.g., due to the extra surface area provided by the second leg 208b of the opened section 204). It should be noted that in FIG. 2C, while the secondary segment of the suture 202 is passed through the second lateral row anchor 104b, the suture itself is still in a slack state, which means that the attached suture is not pulled tight and/or fixed by the second lateral row anchor 104 at this moment.

FIG. 2D illustrates the secondary segment of the suture being pulled tight by tensioning the secondary segment 212b of the suture (e.g., by pulling the free end of the secondary segment). In some embodiments, the fixing of the suture 202 (as described further herein) may include securing the suture to the second lateral row anchor 104b, and/or the first lateral row anchor 104a if the primary segment was not previously secured to the first lateral row anchor 104a. In some embodiments, such securing the suture to the first and/or second lateral row anchor 104a-b includes, for example clamping the suture to the anchor (e.g., as seen with a suture anchor described herein or known in the art). In some embodiments, if the first lateral row anchor 104a has not been secured at the beginning, the first lateral row anchor 104a may be implanted to the surgical site (e.g., humerus bone) and secured first, and then the free end of the secondary segment is pulled tight and fixed into the position by securing the second lateral row anchor 104b. The medial row anchor 102a may be secured before or after the lateral row anchor 104a/104b is secured. In some embodiments, even if the medial row anchor 102a is fixed (e.g. within the humerus bone), the suture 202 may be still in a freely sliding state. In some embodiments, the suture 202 may be also fixed at the second lateral row anchor 104b first, and then the suture 202 is pulled tight and fixed into the position on the first lateral row anchor 104a, which is not limited in the present disclosure.

In some embodiments, when the secondary segment 212b is pulled tight, the opened section 204 may be also pulled tight simultaneously. For example, as shown in FIG. 2D, the loop or opening 206 (“loop” or “opening” may be interchangeably used throughout the application) may be enlarged. In some embodiments, the second leg 208b of the opened section 204 is stretched and pulled away from the first leg 208a via contact with the secondary segment 212b of the suture 202. Accordingly, in some embodiments, the second leg 208b of the opened section 204 provides additional surface area coverage in facilitating repair of the subscapularis repair site.

As described herein, in some cases, the amount of compressive strength applied by the second leg 208b on the subscapularis tendon is adjustable, based at least in part on a distance between the respective medial and lateral row anchors coupled to the second segment (e.g., second lateral row anchor 104b and the medial row anchor 102a), and a length of the second leg 208b. For example, in some embodiments, in order for the opened section 204 to be configured to be pulled tight, a length of the second leg 208b of the opened section 204 may be restricted, such that when the second segment 212b of the suture 202 is pulled tight, the second leg 208b is also pulled tight (e.g., on both sides of a contact point with the secondary segment) without any or with a reduced amount of slack in the second leg 208b. In some embodiments, further tensioning the secondary segment (e.g., pulling the secondary segment) may stretch the second leg 208a, thereby increasing the compressive strength by the second leg 208b about the repair site and/or the surface area coverage by the suture 202. In some embodiments, such tensioning, amount of surface area coverage, and/or compressive strength may be varied based on patient-specific anatomy, among other possible factors. For example, spacing between anchors (which impacts suture net spacing), where a surgeon positions the start site of the suture net, how many legs the surgeon crosses the suture through (which determines the size and/or density of the formed suture net), the amount of tension applied to the suture net, all of which may vary in forming the suture net. This then allows the suture net disclosed herein to be used in variable anatomy sizes, to create variable net footprint, to be used with or without shoulder implant, and/or to be used in open or closed procedures.

In some embodiments, the second leg 208b of the opened section 204 may have a length that is further limited (e.g., as compared to FIG. 2D) such that the secondary segment 212b of the suture 202 does not form a straight configuration between the medial row anchor 102a and the second lateral row anchor 104b when tensioned (e.g., pulled tight), but rather have an inflection point at the contact point (e.g., interweaved point) between the second leg 208b of the opened section 204 and the secondary segment 212b of the suture 202. Accordingly, such further limitation in length of the second leg 208b may help ensure the opened section 204 of the suture 202 is pulled tight when the secondary segment of the suture 202 is pulled tight after passing through the second lateral row anchor 104b.

In some cases, the opening 206 forms an approximately triangular shape when the suture 202 is pulled tight, where a point of contact between the secondary segment 212b and the second leg 208b of the opened section 204 forms a vertex of said triangular shape.

In some embodiments, the length of the second leg 208b of the opened section 204 of the suture 202 varies and depends, at least in part, on a length of the first leg 208a of the opened section, a distance between the first lateral row anchor 104a and the medial row anchor 102a coupled to the suture 202, and/or a distance between the left and second lateral row anchors 104a and 104b.

Toward real applications, a series of sutures 202 having opened sections 204 may be produced or manufactured for the disclosed subscapularis repair systems. The series of bifurcated sutures 202 may have different lengths of the first legs and/or second legs of the opened sections included therein. In actual surgery, a first and/or second leg of an opened section in a suture may be selected based on the size of the tear, body size, and other anatomical features and/or dimensions, among other possible factors that may affect the optimal positions of the anchors for securing the suture.

In some embodiments, different approaches may be utilized to generate a suture having an opened section disclosed herein. In one embodiment, a suture (e.g., 202) may be formed by using two separate threads. For example, the two threads may be tied at two separate middle points with different lengths between the tied points for each thread. That is, one thread may have a larger length between the two tied points when compared to the other thread. In this way, an opened section may be then formed in the suture containing the opened section. In some embodiments, the remaining portions (e.g., the portions before and after the formed opened section) of the two threads may have the same lengths. In some embodiments, the remaining portions of the two threads may be also tied in some way to facilitate anchoring of the formed bifurcated suture.

In some embodiments, a suture 202 with an opened section may be formed through a single thread. For example, current suture and fiber technologies may allow a formation of a bifurcated braid, where a single suture can be divided into two (or even more) separate flat tape strands and then braided back into a single suture or tape segment. Before braiding the flat tape strands back, the two different strands may be braided to different lengths so that an opened section with different leg lengths can be formed after the strands are braided back into a single suture or tape segment.

In some embodiments, an opened section of a suture may be also formed by affixing a separate strand with a proper length to two locations (such as the proximal and distal locations of a to-be-formed opened section) of an existing suture. Different techniques may be utilized for affixing the separate strand, which include but are not limited to tying, braiding, adhesion, and the like, and any combination thereof. In some embodiments, other possible suture-forming technologies may be utilized to form an opened section, which is not limited in the present disclosure.

In some embodiments, to make sure the opened section can hold the stress when the bifurcated suture is pulled tight, the two merge points (e.g., the points indicated by arrows 214 in FIG. 2D) of the opened section may be strengthened by forming a knot or by adding an additional reinforcement structure such as a reinforcement ring or other suitable structures. This may allow the formed opened section to hold the stress better when the suture is pulled tight during the process of fixing the suture by the right and/or first lateral row anchor(s) 104a-b.

It should be noted that, while only one opened section 204 is included in the suture 202 in FIGS. 1-2D, in some embodiments, more than one opened section can be formed along the suture. For example, there may be two or more opened sections 204 formed along the suture.

The two or more opened sections 204 included therein may have different loop sizes (e.g., different sized openings 206) from each other. For example, the formed loops or openings closer to the lateral row anchor 104a may have a larger size. For example, the second leg 208b of an opened section 204 closer to the lateral row anchor 104a may have a larger length than the second leg of an opened section closer to the medial row anchor 102a. In some embodiments, the two or more opened sections are all located in the primary segment 212a of the suture (i.e., between the first lateral row anchor 104a and the medial row anchor 102a).

It should be also noted that while the opened section 204 is shown to be located at a portion of the suture between the first lateral row anchor 104a and the medial row anchor 102a (i.e., the primary segment of the suture), in some embodiments, the opened section 204 may be located at the secondary segment of the suture instead. In such circumstances, in some embodiments, to form a suture net, the second end of the suture 202 may be secured to the second lateral row anchor 104b first before being secured to the first lateral row anchor 104a.

The first end of the bifurcated suture 202 then passes through the loop 206 of the opened section (on the secondary segment) before passing through and being pulled tight and secured to the first lateral row anchor 104a. In some embodiments, multiple opened sections may be also formed in the secondary segment of the suture, similar to the situations where there are multiple opened sections in the primary segment of the suture described above. Accordingly, in some embodiments, any number of opened sections can be located on the primary segment 212a, the secondary segment 212b, or both, of the suture.

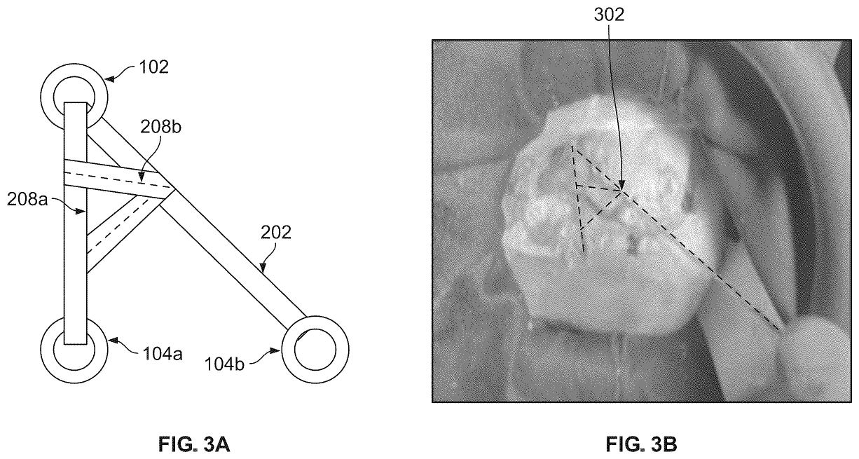

FIG. 3A further illustrates an exemplary suture net formed through the process described in FIGS. 2B-2D, wherein as depicted, a larger distance between the two lateral row anchors will vary the tensioned configuration of the opened section, specifically the second leg 208b (for example as compared with FIG. 2D). FIG. 3B illustrates an image of an exemplary application of using a suture net on a soft tissue. The suture net shown in FIG. 3A is similar to a suture net shown in FIG. 1, details of which are not repeated here. The suture net shown in FIG. 3A is included here mainly as a reference to show how such a suture net can be actually applied in real applications as shown in FIG. 3B. Specifically, as illustrated in FIG. 3B, a suture net may be implanted during surgery at or in place of a damaged soft tissue. The suture net may be formed therein and/or deployed into the damaged site of the soft tissue in a guided or direct manner. For example, the above-described medial row anchor and/or lateral row anchors or other types of anchors may be utilized in forming a suture net at or in place of the damaged soft tissue. The formed suture net (e.g., suture net 302 shown in FIG. 3B, which is pulled tight but has not been fixed by the second lateral row anchor yet) may have a similar structure as shown in FIG. 3A.

In some embodiments, any suitable suture anchor may be used as a lateral row anchor and/or medial row anchor for securing the suture net (e.g., to a bone, tissue, etc.), depending upon the needs of the damaged soft tissue being repaired. In some embodiments, a suture anchor used herein may be a screw or threaded anchor or an impaction or threadless anchor based on the way an anchor is fixed to the target site (e.g., a target bone). In some embodiments, a suture anchor used herein may also be classified based on how sutures are tightened, typically, as normal anchors or knotless anchors. Knotless anchors include an eyelet-suture system rendering knot-tying unnecessary. In some embodiments, an anchor used herein may be categorized based on material. For example, a suture anchor used herein may be formed from bioinert and biocompatible metals such as titanium, stainless steel and alloys thereof. In some embodiments, a suture anchor used herein is made of a bioabsorbable material or combination. Exemplary materials include PGA, polylactic acid (PLA) a copolymer of PLA and PGA (PLGA) or a combination of the two. Additional alternatives for a suture anchor used herein include anchors that are biocomposites having a bioabsorbable polymer and an osteoconductive bioceramic or such materials in combination to accelerate bone formation and mineralization by the timed degradation of the ceramic along with the polymer. Still another alternative suture anchor material is PEEK. In some embodiments, an all-suture anchor is used for any of the medial row and lateral row anchors. In some embodiments, for any anchor used herein for a medial and/or lateral row anchor, the anchor may comprise a wedge push-in suture anchor (e.g., as described in U.S. Pat. No. 11,298,120, which is incorporated herein by reference in its entirety), a large push-in suture anchor (e.g., as described in PCT application PCT/US2021/045406, which is incorporated herein by reference in its entirety), an all-suture anchor (e.g., as described in PCT application PCT/US2022/075143, which is incorporated herein by reference in its entirety), other types of push-in anchor (e.g., as described in PCT application PCT/US2022/076334, which is incorporated herein by reference in its entirety), or any combination thereof.

It should be noted that while one exemplary application of the disclosed suture net to a damaged soft tissue (e.g., subscapularis tears) is illustrated in FIG. 3B, in real applications, a suture net may be formed in a similar manner during any of a wide variety of surgical procedures such as an open procedure, a “mini-open” procedure, a minimally invasive surgical procedure, a natural orifice transluminal surgical procedure, a single port access procedure, an endoscopic procedure, an arthroscopic procedure or any other scope based procedure.

Referring back to FIG. 1, while only one suture net is illustrated in the figure, in some embodiments, more than one suture net may be formed in real applications. For example, in some embodiments, the suture 108 in FIG. 1 may be also a suture having an opened section, which then allows a suture net to be formed in a same way as that formed by the suture 202.

In addition, in some embodiments, a second suture net may be further formed between the two lateral row anchors 104a and 104b and the medial row anchor 102a shown in FIG. 2C. For example, another suture having an opened section may be used to form such a second suture net by disposing the opened section of the suture between the medial row anchor 102a and the second lateral row anchor 104b shown in FIG. 2C (this embodiment is however not shown in FIG. 2C). For example, a second suture may provide a mirror image version of the suture net depicted FIGS. 2C-D. In some embodiments, for such an embodiment, the first suture (e.g., 202) and/or second suture may be configured to pass through the opened sections of both sutures, thereby being interweaved together.

It should be noted that the positions of the lateral row anchors and medial row anchors shown in FIG. 1 are merely for illustrative purposes but not for limitations. For example, in real applications, a distance between the two lateral row anchors 104a and 104b may be different from a distance between two medial row anchors 102a and 102b, and/or a distance between the first lateral row anchor 104a and medial row anchor 102a may be different from a distance between the second lateral row anchor 104b and medial row anchor 102b. In addition, the orientation of the overall structure shown in FIG. 1 may also vary and be different from that shown in FIG. 1, depending on the surgical site in real applications. In addition, while two medial row anchors 102a and 102b are illustrated in FIG. 1, in real applications, as described herein, there may be fewer (e.g., one) or more (e.g., three or more) medial row anchors in real applications. In some embodiments, the number of lateral row anchors may also vary in real applications. This provides additional adjustability for variable anatomy and spacing of anchors.

It should be also noted that the above-described formation of a suture net is just one example of forming a suture net at a surgical site. In real applications, many other different means for forming a suture net may be also possible, as long as the formed suture nets can be spread across (e.g., tensioned configuration) and be taut despite different coverage areas for different patients.

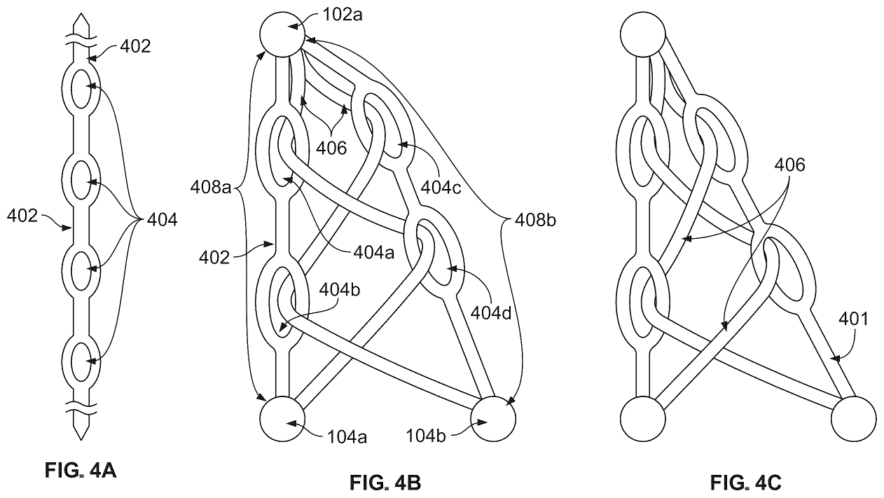

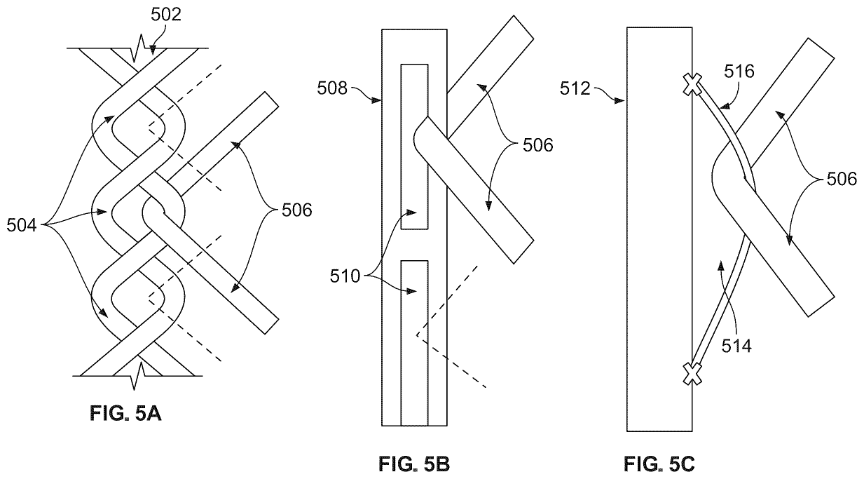

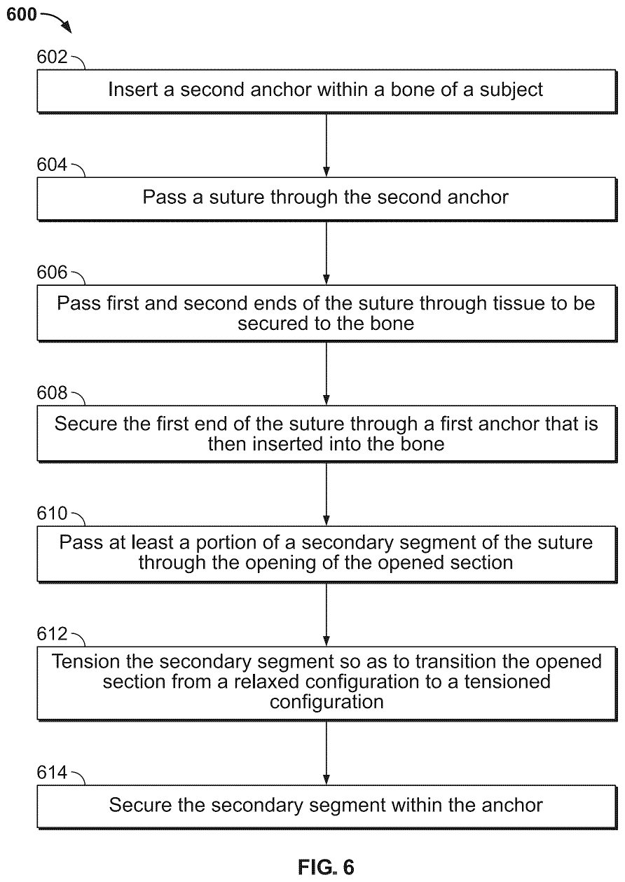

In the following, another exemplary approach for forming a suture net is further described. Compared to the suture net formed based on a suture (e.g., 202) described above, the suture net described below, in some embodiments, is formed like a shoelace, where a limb suture forms an interweaved net structure by passing through separate rows of loops (e.g., openings) that are defined by a separate suture. The as-formed suture net may also allow for free sliding and may be adjustable, as will be described more in detail below with reference to FIGS. 4A-5C.

FIGS. 4A-4C illustrate an exemplary process for forming a shoelace type suture net. Specifically, FIG. 4A illustrates a multi-opened section suture (e.g., suture tape) 402 that includes a plurality of loops (or openings) 404 aligned along the suture tape. The suture openings 404 may vary in size as compared to the opening 206 shown in FIG. 2A (e.g., some openings 404 may be larger, smaller, or same size as opening 206). In some embodiments, the multi-opened section suture tape 402 is secured to a medical row anchor 102a shown in FIG. 4B. The multi-opened section suture tape 402 may be passed through the medical row anchor 102a at approximately a middle point, as indicated by the middle point 406 in FIG. 4A. At this time point, the suture 402 may freely slide around the medial row anchor 102a. In some embodiments, when the multi-bifurcated suture 402 is secured by the medial row anchor 102a, a separate suture limb with or without any opening (e.g., 406 which is without any opening) may be similarly passed through the medial row anchor 102.

In some embodiments, the two free ends of the suture 402 may be further passed through the first lateral row anchor 104a and second lateral row anchor 104b. The suture 402 may thus form two segments, a first segment (or primary segment) 408a between the medial row anchor 102a and the first lateral row anchor 104a, and a second segment (secondary segment) 408b between the medial row anchor 102a and the second lateral row anchor 104b. Each segment includes a number of loops or openings 404, as shown in FIG. 4B.

In some embodiments, after the suture limb 406 is passed through the medial row anchor 102a, the two free ends of the suture 406 are passed through the openings 404 or loops in the primary segment and the secondary segment 408b of the suture 402 in a manner like tying a shoelace. For example, for a first end of the suture limb 406, it may be passed through a loop 404a and then a loop 404d. For a second end of the suture limb 406, it may be passed through a loop 404c and then a loop 404b, as shown in FIG. 4B. Each of the first end and the second end of the suture limb 406 may be then secured to the first lateral row anchor 104a or the second lateral row anchor 104b, respectively. In this way, a suture net interweaved structure can be formed by the suture limb 406, as shown in FIG. 4B. At this stage, the formed suture net as a whole may be still in a slack state. That is, the suture tape 402 and/or the suture limb 406 are not pulled tight at this stage.

FIG. 4C shows a stage when the suture tape 402 and the suture limb 406 are pulled tight and fixed to the first lateral row anchor 104a and the second lateral row anchor 104b. As can be seen, compared to the suture net shown in FIGS. 1-2D, a suture net formed through the process shown in FIGS. 4A-4C has an increased suture surface area and likely an increased compression on the subscapularis tendon at a surgical site (interchangeably referred to repair site herein).

It should be noted that, while the suture limb 406 is shown to pass through the medial row anchor 102a, in some embodiments, the suture limb 406 does not necessarily pass through the medial row anchor 102a. In one example, one end of the suture limb 406 may sequentially pass through the first lateral row anchor 104a, the loops 404d, 404a, 404c, 404b, then the second lateral row anchor 104b. In another example, one end of the suture limb 406 may sequentially pass through the second lateral row anchor 104b, the loops 404b, 404c, 404a, 404d, and the first lateral row anchor 104a. Alternatively, a first end of the suture limb 406 sequentially passes through the loops 404a, 404d, and the first lateral row anchor 104a, while a second end of the suture limb 406 sequentially passes through the loops 404c, 404b, and the second lateral row anchor 104b. In some embodiments, other ways of forming a shoelace type suture net without passing through the medial row anchor 102a are also possible and contemplated by the disclosure.

It should be also noted that while two loops are illustrated in each of the two segments of the suture tape 402 in FIGS. 4B-4C, the exact number of loops included in each segment may vary. In general, a larger number of loops in each segment may increase a suture surface area and/or compression force about the subscapularis tendon when these loops are all used for holding one or more interweaved suture limbs (e.g., 406). In addition, in some embodiments, there is a same or different number of loops in the two segments (primary and secondary) although the exact number may vary. In some embodiments, the distance between adjacent loops in the two segments may be different, since the two segments may have different lengths but have the same number of loops, as shown in FIG. 4B. For example, the distance between adjacent loops in the segment 408b may be larger than that of the segment 408a.

In some embodiments, if there are more than two loops in a given suture (e.g., 402), the distance between two adjacent loops in each segment may also vary. For example, the distance between two adjacent loops may be larger when these loops are closer to the medial row anchor 102a and smaller when these loops are closer to the lateral row anchors 104a-b. This may improve the even distribution of the interleaved suture limb since the lower portion of the interweaved suture limb may be wider, as shown in FIGS. 4B-4C.

In some embodiments, the loops 404 in each suture tape 402 may also have different sizes. In one embodiment, the loops 404 in each segment of the suture tape 402 may be small and mainly used for holding the suture limb 406 when forming the shoelace type suture net. Under such circumstances, the two legs of these loops 404 may have a same length or approximately the same length. In an alternative embodiment, one or more loops in each segment of the suture tape 402 may be larger than others and the opened sections may have uneven legs as described in FIG. 1 (e.g., the second leg of an opened section is longer than the first leg of the opened section), although these loops may be still considerably smaller than the loops shown in FIGS. 1-2D. In other words, these loops may allow to form a triangle shape or approximately a triangle shape when being pulled tight from the ends of the multi-bifurcated suture tape 402 and the suture limb 406 (e.g., by pulling the suture tape 402 and/or the suture limb 406), but the formed triangle shapes by these loops 404 still may not reach to the triangle shape formed from a corresponding loop in the other segment of the multi-bifurcated suture tape.

In some embodiments, the as-formed suture net may be referred to as a “hybrid” suture net, as it combines some features of suture net shown in FIGS. 1-2D and some features of shoelace type suture net shown in FIGS. 4B-4C. Although not shown, a hybrid suture net may have three different regions, a left region formed by the primary segment of the suture tape 402, an interweaved suture limb region in the middle, and a right region formed by the secondary segment of the suture tape 402. In some embodiments, not both the primary and secondary regions need to be present in a hybrid suture net. For example, a hybrid suture net may only include a left region and a middle region, or only include a right region and a middle region.

In some embodiments, to allow a suture net to be even distributed, for a hybrid suture net, the loop sizes of different loops may also vary. For example, for the loops closer to the lateral row anchors 104, the loops may become larger. Accordingly, for a hybrid suture net that includes three regions as described above, the left and right regions may have an overall triangle shape (e.g., having wider areas close to the lateral row anchors 104 and the narrow areas close to the medial row anchor 102), while the middle region may have an overall trapezoid shape or another irregular or regular polygon shape.

It should be noted that while only one shoelace type suture net is illustrated in FIGS. 4B-4C, in some embodiments, there may be more than one shoelace type suture net or hybrid suture net formed at a surgical site. For example, there may be an additional medial row anchor, which allows the formation of a second shoelace type suture net or hybrid suture net (e.g., by using one or more additional sutures having opened sections and/or one or more additional suture limbs). The two shoelace type suture nets and/or hybrid suture nets may share the two lateral row anchors shown in FIGS. 4B-4C. In some embodiments, there may be more than two medial row anchors and/or more than two lateral row anchors, and thus more than two shoelace type suture nets and/or hybrid suture nets may be formed at a surgical site. In real applications, these shoelace type suture nets and/or hybrid suture nets may also have various sizes and orientations, depending on the actual needs of the real applications.

In some embodiments, other additional types of loops (openings) may be utilized with a suture having an opened section, as described herein, and for example, with use in forming any repair system described herein. FIGS. 5A-5C show exemplary types of opened sections that can be used in any type of repair system described herein, including a shoelace type suture net formation. Specifically, FIG. 5A shows an exemplary multi-opened section 502 that includes a row of loops 504 aligned along the multi-opened section. The multi-opened section 502 shown in FIG. 5A is very similar to the suture tape 402 shown in FIG. 4A, except that the density of loops in FIG. 5A is higher. In some embodiments, when the density of loops is high, it may provide flexibility in forming the suture net. For example, when the density of loops is high, not every loop may be used in holding a suture limb 506 in forming a suture net. Accordingly, when forming a suture net, a loop in a desired location may be selected to allow the formation of a suture net with a more evenly distributed net structure. In some embodiments, the loops 504 in the multi-opened section 502 may be formed by tying two different threads at multiple locations along the two threads, which leaves the openings between adjacent tying points. In some embodiments, the loops 504 may be formed through a braiding process. Other processes for forming the loops in FIG. 5A may be also possible and are contemplated in the present disclosure.

FIG. 5B shows another exemplary multi-opened section 508, according to some embodiments. As shown in FIG. 5B, the multi-opened section 508 includes multiple narrow long openings 510 along the multi-opened section. A long opening 510 may allow an attached suture limb (e.g., suture limb 506) to freely slide within the opening, and thus provide the flexibility in suture net formation, e.g., by allowing the suture limb 506 to freely slide to pinpoint to an optimal location in the opening when the suture limb 506 and/or the bifurcated suture is pulled tight.

FIG. 5C shows another exemplary straight suture 512 used in forming a suture net. As can be seen, instead of forming a loop or opening within a bifurcated suture as shown in FIGS. 5A-5B, a loop 514 may be formed on one side of the straight suture 512. The loop 514 may be formed by tying or braiding a separate strand 516 at two ends, as shown in FIG. 5C. A suture limb 506 may then pass through the loop 514 when forming a suture net. In the loop structure shown in FIG. 5C, the suture limb 506 may also freely slide, thereby increasing the flexibility in forming a suture net. In addition, without openings in the straight suture 512, the suture 512 may hold a larger stress when being pulled tight. The separate strand 516 for forming the loop 514 may have a higher strength than the straight suture 514, and thus may withhold a same pulling force even though the strand 516 has a much smaller width or diameter than that of the straight suture 512. This type of loop may be useful if the stress withheld by a bifurcated suture needs to be very high (e.g., damage is more serious), and thus a bifurcated suture with loops inside the suture as shown in FIGS. 5A-5B may be not sufficient to withhold the stress required in real applications.

In some embodiments, there may be additional types of loops used in suture net formation, which is not limited in the present disclosure. These various loops may provide great flexibility in forming shoelace type suture nets, hybrid suture nets, or other types of suture nets not described above, as long as these suture nets are able to lay flat and be taut despite different coverage areas for different patients.

Referring now to FIG. 6, an exemplary method 600 for forming a suture net using a suture anchor system disclosed herein is further described. The suture net may correspond to a suture net shown in FIGS. 1-2D.

At step 602, a second anchor is inserted within a bone of a subject. In one example, the subject is a human, and the bone may be the humerus. In some embodiments, in the suture anchor system disclosed herein, there may be a plurality of anchors, where each anchor is configured to be independently inserted within a respective location of a tissue or bone of the subject about a repair region thereof. The plurality of anchors may include a first, second, and third anchor that allow to form a suture net. The second anchor may be a medial row anchor (e.g., medial row anchor 102 as shown in FIGS. 2B-2C) while the first anchor and the third anchor may be lateral row anchors (e.g., medial row anchors 104a and 104b shown in FIGS. 2B-2C).

At step 604, a suture is passed through the second anchor. In the embodiments disclosed herein, the suture may be a suture having an opened section for forming a suture net as described in FIGS. 1-2D (e.g., 202). In some embodiments, the suture is not yet fixed within the second anchor (e.g., the medial row anchor 102a as shown in FIGS. 2B-2C), such that the suture remains free sliding (e.g., for tensioning). In other cases, the suture may be fixed and substantially immobilized by the second anchor. In some embodiments, when the suture passes through the second anchor, one end of the suture may pass through the second anchor and pause when a certain length of the suture has passed through the second anchor. Accordingly, two ends of the suture with certain lengths may extend from the second anchor.

At step 606, first and second ends of the suture are passed through a soft tissue to be secured to the bone, such that the first and second ends of the suture passes through one or more apertures of the soft tissue (e.g., subscapularis tendon) from a bone facing portion of the soft tissue to an opposite facing portion of the soft tissue. Here, the segment of the suture from the first end to the aperture defines the primary segment, comprising an opened section (e.g., an opened section 204 shown in FIG. 2A) having a first leg (e.g., the left leg 208a), a second leg (e.g., the second leg 208b in FIG. 2A), and an opening (e.g., opening 204 in FIG. 2A) therebetween.

At step 608, the first end of the suture is passed through the first anchor that is then inserted into the bone. Here, the first anchor may be a lateral row anchor (e.g., first lateral row anchor 104a shown in FIG. 2C). The primary segment may be thus also defined as a segment between the first anchor and the second anchor, according to some embodiments. In some embodiments, the anchor is inserted within the bone first, and the suture is passed therethrough. In some embodiments, the suture may not yet be fixed within the anchor, so as to allow the suture to be tensioned. In some embodiments, the suture can be fixed within the anchor.

At step 610, at least a portion of a secondary segment of the suture is passed through the opening of the opened section, and then through a third anchor that is then inserted into the bone, the secondary segment comprising the suture from the second end to the aperture. In some embodiments the third anchor is inserted within the bone prior to have the second inserted therethrough. In some embodiments, as shown in FIG. 2B, the second end of the suture may pass through the opening of the opened section, to interweave with the primary segment of the suture (e.g., the second leg 208b of the opened section as shown in FIG. 2B). The second end of the suture may be then passed through the third anchor, which may be a second lateral row anchor (e.g., the second lateral row anchor 104b as shown in FIG. 2C). The secondary segment may be defined as a segment between the aperture and the second end or a segment of the suture between the second anchor and the third anchor according to some embodiments (e.g., segment 212b shown in FIG. 2C).

At step 612, the secondary segment is tensioned so as to transition the opened section from a relaxed configuration to a tensioned configuration via interaction between the secondary segment and the second leg, such that a compressive force is applied about the tissue on the bone by the primary segment, the secondary segment, and the second leg. In some embodiments, after the second end of the suture passes through the third anchor, a suture net is formed as the second segment interweaves with the opening in the opened section of the suture. The suture net may be still in a slack or relaxed configuration. Through the tensioning of the secondary segment, the suture net may be tensioned since the secondary segment interweaves with the second leg of the opening in the opened section. When the suture net is tensioned, a compression force may be applied about the tissue on the bone by the suture net, including the primary segment, the secondary segment and the second leg.

At step 614, the secondary segment is fixed within the third anchor (e.g., via a clamping feature). In some embodiments, when tensioning the secondary segment, the first anchor may be already fixed, and thus the first end of the suture does not slide along the first anchor. The second anchor may be also secured, but the suture itself can still freely slide along the second anchor. In some embodiments, the suture may also be fixed within the second anchor. Accordingly, at this step, securing the suture net may include securing the secondary segment of the suture via the third anchor. In some embodiments, the suture net may be also secured by fixing the suture net at the second anchor, depending on the configuration of the second anchor.

It should be noted that, while a single opened section is included in the suture, in some embodiments, one or more additional opened sections (e.g., one or more additional opened sections shown in FIG. 2B) may be included in the suture. When there are multiple opened sections in the primary segment, the secondary segment of the suture may pass through each corresponding before passing through the third anchor.

It should be also noted that, the steps 602-614 described above are for securing one suture or suture net. In some embodiments, a second suture net may be similarly secured, as described earlier in FIG. 2. For example, in some embodiments, the suture anchor system disclosed herein further includes a second suture that contains a third end, a fourth end, and a second opened section therebetween. The second opened section defines a third leg, a fourth leg and a second opening therebetween. The second suture is configured to pass through a fourth anchor (e.g., the right medial row anchor 102b shown in FIG. 1) and then the third and fourth ends of the second suture pass through another different aperture of the soft tissue. The third end of the second suture can be then secured to the third anchor (e.g., the second lateral row anchor 104b shown in FIG. 1), while the fourth end of the suture may pass through the second opening of the second suture before being secured to the first anchor (e.g., the first lateral row anchor 104a). Tensioning the fourth end of the second suture may also transition the second opened section from a relaxed configuration to a tensioned configuration via interaction between the different segments and the legs of the second suture, such that a compressive force is applied about the tissue on the bone by the formed suture net.

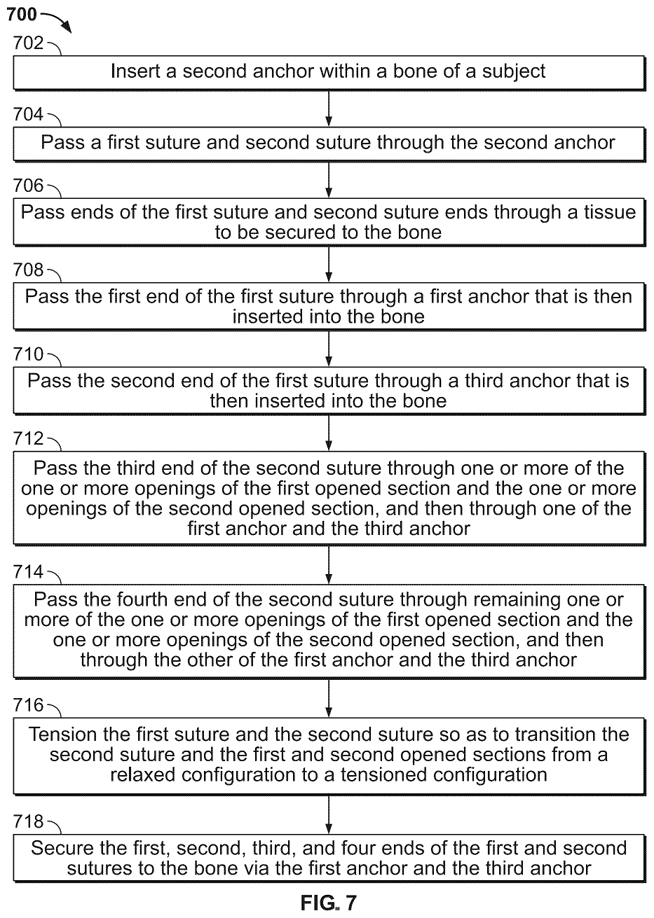

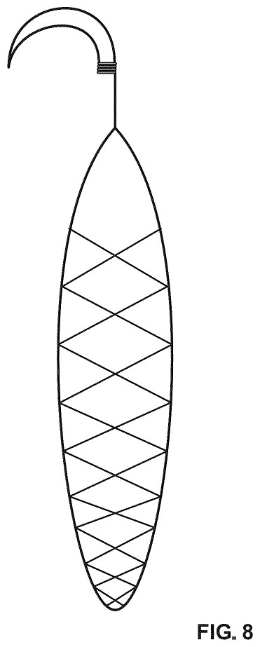

Referring now to FIG. 7, another exemplary method 700 for forming a suture net using a suture anchor system disclosed herein is described. The suture net may correspond to a shoelace suture net shown in FIGS. 4A-4C.

At step 702, a second anchor is inserted within a bone of a subject. In some embodiments, the subject may be a human, and the bone may be humerus. The second anchor may be a medial row anchor (e.g., the medial row anchor 102a shown in FIG. 4B).

At step 704, a first suture and second suture are passed through the second anchor, the first suture having a first and second end extending from the second anchor, the second suture having a third and fourth end extending from the second anchor. According to some embodiments, the first suture may be a suture having two or more opened sections that includes a plurality of the loops (e.g., openings) aligned along the suture (e.g., loops 404 shown in FIG. 4A). The second suture may be a suture limb that does not include loops (e.g., 406 in FIG. 4B). It should be noted that, in some embodiments, the second suture may not pass through the second anchor. That is, when forming a suture net, the second suture may pass through the loops of the first suture without necessarily passing through the second anchor (e.g., without passing through the medial row anchor 102a shown in FIG. 4B).

At step 706, the first, second, third, and fourth ends are passed through a soft tissue to be secured to the bone, such that the first suture and the second suture passes from a bone facing portion of the soft tissue (e.g., subscapularis tendon) and through an aperture to an opposite facing portion. In some embodiments, the first, second, third and fourth ends may pass through a same aperture of the soft tissue. In some embodiments, only the first and second ends of the first suture pass through the aperture if the second suture does not pass through the second anchor as described above at step 704.

At step 708, the first end of the first suture is passed through a first anchor that is then inserted into the bone (or alternatively, the first anchor is first inserted into the bone before having the suture passed therethrough), where the first suture from the first end to the aperture defines a primary segment comprising a first opened section having one or more openings therebetween. According to some embodiments, the first anchor may be a first lateral row anchor 104a as shown in FIG. 4B. The primary segment may be a segment 408a as shown in FIG. 4B.

At step 710, the second end of the first suture is passed through a third anchor that is then inserted into the bone (or alternatively, the third anchor is first inserted into the bone before having the suture passed therethrough), where the first suture from the second end to the aperture defines a secondary segment comprising a second opened section having one or more openings. According to some embodiments, the third anchor is the second lateral row anchor 104b as shown in FIG. 4B. The secondary segment may be segment 408b shown in FIG. 4B.

At step 712, the third end of the second suture is passed through one or more of the one or more openings of the first opened section and the one or more openings of the second opened section, and then through one of the first anchor and the third anchor. According to some embodiments, the third end of the second suture may pass through one or more of the one or more openings of the first opened section and the one or more openings of the second opened section in a manner similar to tying a shoelace. For example, the third end of the second suture may pass through a first opening on the primary segment, a second opening on the secondary segment, a third opening on the primary segment, a fourth opening on the secondary segment, and so on, until the third end of the second suture pass through the last opening on one of the primary and secondary segments. Alternatively, the third end of the second suture may pass through the first opening on the secondary segment, second opening on the primary segment, third opening on the secondary segment, fourth opening on the primary segment, and so on, until the third end of the second suture passes through the last opening on one of the primary and secondary segments. Here, the order of the openings in each of the primary and secondary segments is numbered according to a distance to the medial row anchor or the aperture of the tissue. For example, an opening closest to the medial row anchor or the aperture of the tissue is the first opening, while an opening furthest away from the medial row anchor or the aperture of the tissue is the last opening on each of the primary and secondary segments.

At step 714, the fourth end of the second suture is passed through the remaining one or more of the one or more openings of the first opened section and the one or more openings of the second opened section, and then through the other of the first anchor and the third anchor (whichever the third end of the second suture did not pass through). In some embodiments, the fourth end of the second suture passes through the remaining openings of the one or more openings in the first opened and second opened sections in a similar manner as step 712. For example, if the third end of the second suture has passed through the first opening of the primary segment, the fourth end of the second suture then passes through the first opening of the secondary segment, the second opening of the primary segment, the third opening of the secondary segment, and so on, until the fourth end of the secondary segment reaches the last opening of the primary or secondary segment. It should be noted that if the third end of the second suture has passed through the last opening on the primary segment, the fourth segment of the second suture will pass the last opening on the secondary segment, and vice versa.

At step 716, the first suture and the second suture are tensioned so as to transition the second suture and the first and second opened sections from a relaxed configuration to a tensioned configuration via interaction between the second suture and the one or more openings in the first opened sections and the one or more openings in the second opened section, such that a compressive force is applied about the tissue on the bone by the first suture and the second suture. In some embodiments, after the third end and the fourth end of the second suture pass through the first and third anchors, a shoelace type suture net is formed but not tightened yet. That is, the shoelace type suture net is in a related or slack state, as shown in FIG. 4B. Accordingly, the first suture and the second suture can be tensioned to transition the suture net from the relaxed configuration to the tensioned configuration.

At step 718, the first and second sutures are secured to the bone via the first anchor and the third anchor (e.g., the first and second sutures are fixed to the first and third anchors). In some embodiments, one of the first anchor and third anchor may be fixed first before tensioning the first suture and the second suture through the other of the first anchor and the third anchor. In some embodiments, the first suture and the second suture can freely slide around the second anchor when the first and/or second suture is being tensioned and fixed by the first or third anchor. In some embodiments, if one or both of the first suture and the second suture are found not sufficiently tensioned, the first and/or third anchor may be loosened so that the first suture and/or the second suture can be re-tensioned and fixed again.

Although not illustrated in FIGS. 6-7, in some embodiments, a hybrid suture net or another different type of suture net described above may be similarly applied to a soft tissue and bone.

In some embodiments, the suture nets described above may be formed on site, e.g., a healthcare practitioner may guide an end of a suture to pass through the anchors and/or the loops, and then pull the suture(s) tight before securing the anchors.

In some embodiments, a suture net may be formed in advance and delivered to a surgical site in a pre-formed suture net format. At this moment, to allow the suture net to be fixed through different anchors (e.g., medial row anchor and/or lateral row anchors) at the desired positions, a suture net may further include one or more additional loops disposed at the desired position(s). Thereafter, a suture net can be fixed to an anchor through the additional loop(s). In one example suture net shown in FIGS. 2C-2D, a loop may be added to a bifurcated suture at a position corresponding to the position of the suture where the suture is fixed by the medial row anchor 102 as shown in FIG. 2C. In some embodiments, ends of a pre-formed suture net may still remain free, and thus can be secured and/or fixed to the lateral row anchors in a similar way as a suture net formed on site.

FIG. 8 illustrates an exemplary suture net formed in advance. The suture net in FIG. 8 is a shoelace type suture net, but the pre-formed suture net can be other forms of suture nets described above. In FIG. 8, the ends of the pre-formed suture net are tied for easier handling during packaging and/or delivery, but the ends can be cut to release them for subsequent fixing to the respective lateral row anchors. The suture net formed in advance may save the time required during a surgery, and avoid interweaving of the suture net in a limited space of a surgical site. In addition, the consistency of suture nets can be improved when these suture nets are formed in advance under a controlled environment. It should be also noted that even if these suture nets are formed in advance, these pre-formed suture nets still provide necessary adjustability for variable anatomy and spacing of anchors. For example, these pre-formed suture nets may still be able to lay flat and be taut despite different coverage areas for different patients.

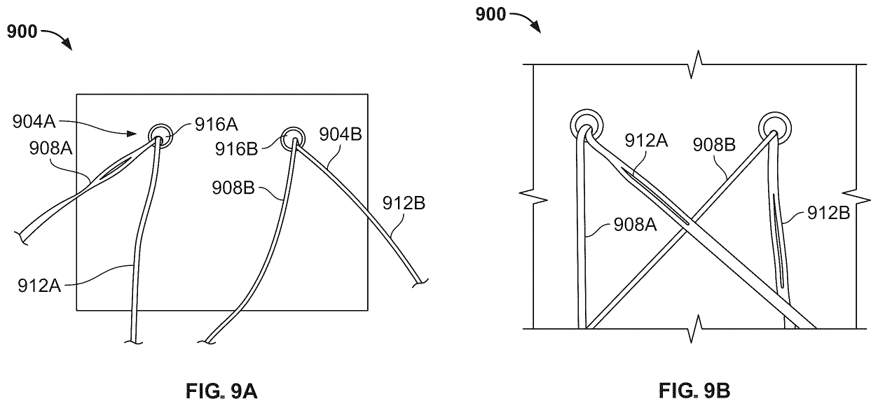

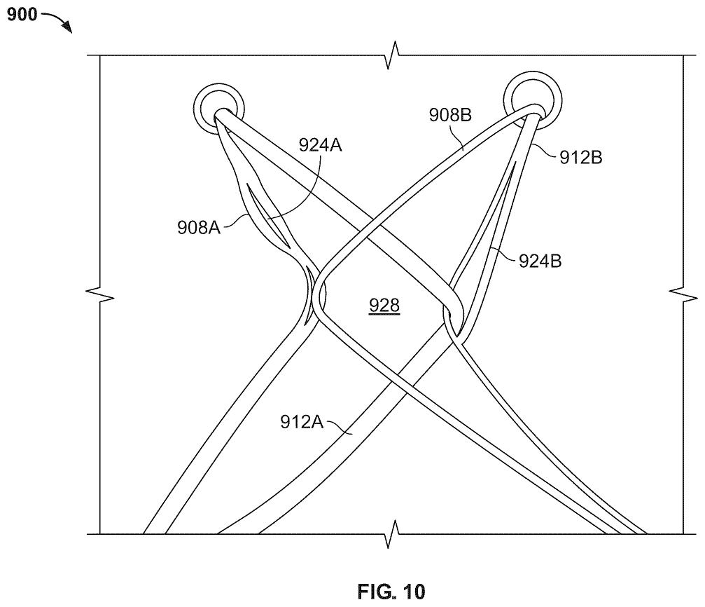

Referring now to FIG. 9A, a suture system 900 is presented. Suture system 900 may include first bifurcated suture 904A and/or second bifurcated suture 904B. First bifurcated suture 904A may have first limb 908A and/or second limb 912A. First and second limbs 908A and 912A may extend from an origin of first bifurcated suture 904A. An origin of first bifurcated suture 904A may be first suture anchor 916A. First suture anchor 916A may be inserted into a first bone location of a subject. First and second limbs 908A and 912A of first bifurcated suture 904A may pass through a soft tissue of a subject from an outside of the soft tissue to an inside of the soft tissue with respect to the subject. Likewise, second bifurcated suture 904B may have first limb 908B and/or second limb 912B. First and second limbs 908B and 912B, of second bifurcated suture 904B may extend from an origin point, such as, but not limited to, second suture anchor 916B that may be inserted into a second bone location of a subject. In some embodiments, first bifurcate suture 904A may be connected to and/or a part of first suture anchor 916A. Likewise, second bifurcated suture 904B may be connected to and/or a part of second suture anchor 916B.

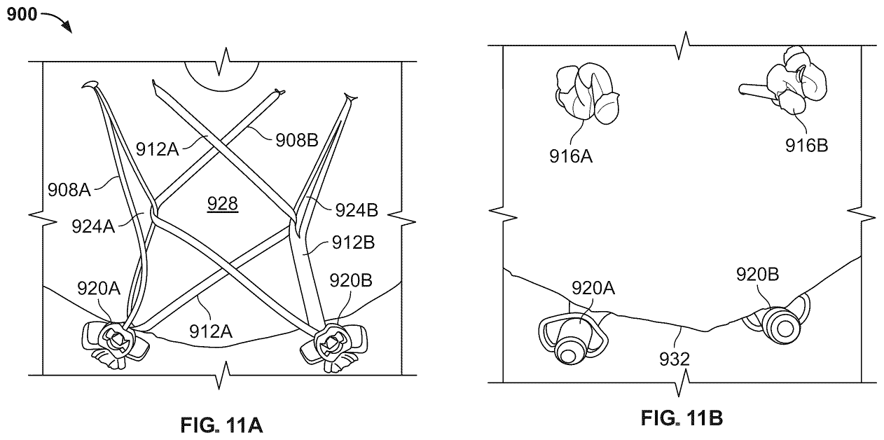

Referring no to FIG. 9B, a weaving of a first and second bifurcated suture is presented. First limb 908A of first bifurcated suture 904A may pass towards a first anchor insert 920A (e.g. as shown in FIGS. 11A, 11B). First anchor insert 920A may be inserted into a first bone location of a subject. In some embodiments, first anchor insert 920A is positioned adjacent to a soft tissue in which a first and/or second suture insert are positioned (e.g. 916A, 916B). First limb 908A of first bifurcated suture 904A may pass in a substantially straight orientation towards first anchor insert 920A. For instance, and without limitation, first anchor insert 920A may be positioned across from a first suture anchor insert of which first bifurcated suture 904A may originate from. In some embodiments, first anchor insert 920A is aligned with a first suture anchor insert across a horizontal axis. Second limb 912A of first bifurcated suture 904A may pass towards second anchor insert 920B. Second anchor insert 920B may be positioned across from a second suture anchor insert, of which second bifurcated suture 904B may originate from. Second limb 912A of first bifurcated suture 904A may pass towards second anchor insert 920B at an angle with respect to a first suture anchor insert. For instance, and without limitation, second limb 912A may pass towards second anchor insert 920B at an angle with respect to a first suture anchor insert of about 10 degrees, about 15 degrees, about 20 degrees, about 25 degrees, about 30 degrees, about 35 degrees, about 40 degrees, about 45 degrees, about 50 degrees, about 55 degrees, about 60 degrees, about 65 degrees, about 70 degrees, about 75 degrees, or greater than about 75 degrees.

Still referring to FIG. 9B, first limb 908B of second bifurcated suture 904B may pass towards first anchor insert 920A. First anchor insert 920A may be inserted into a first bone location of a subject. In some embodiments, first anchor insert 920A is positioned across a first suture anchor insert. First anchor insert 920A may be positioned at a distance from a first suture anchor insert equal to that of a distance between second anchor insert 920B and a second suture anchor insert. In some embodiments, a first suture anchor insert and a second suture anchor insert may be spaced at a distance equal to a distance between first anchor insert 920A and second anchor insert 920B. First limb 908B of second bifurcated suture 904B may pass over or under second limb 912A of first bifurcated suture 904A. For instance, second limb 912A of first bifurcated suture 904A may pass towards second anchor insert 920B. First limb 908B of second bifurcated suture 904B may pass towards first anchor insert 920A before or after second limb 912A of first bifurcated suture 904A passes towards second anchor insert 920B. First limb 908B may pass towards first anchor insert 920A at an angle with respect to a second suture anchor insert. For instance, first limb 908B may pass towards first anchor insert 920A at an angle with respect to a second suture anchor insert of about 10 degrees, about 15 degrees, about 20 degrees, about 25 degrees, about 30 degrees, about 35 degrees, about 40 degrees, about 45 degrees, about 50 degrees, about 55 degrees, about 60 degrees, about 65 degrees, about 70 degrees, about 75 degrees, or greater than about 75 degrees.

A passing or weaving of second limb 912A of first bifurcated suture 904A with first limb 908B of second bifurcated suture 904B may form a diagonal or “crisscross” pattern across a surface of a soft tissue. In some embodiments, system 900, as illustrated in FIG. 9B, may have a horizontal and/or vertical symmetry across a horizontal or vertical axis. For instance, and without limitation, second limb 912A of first bifurcated suture 904A and/or first limb 908B of second bifurcated suture 904B may each pass across a surface of a soft tissue at an angle of about 40 degrees to about 50 degrees. In some embodiments, both second limb 912A and first limb 908B may pass at an angle of about 45 degrees with respect to a first suture anchor insert and a second suture anchor insert, respectively.

Referring now to FIG. 10, an embodiment of a bifurcated suture system of the bifurcated sutures described in FIGS. 9A-B is presented. First bifurcated suture 904A may have first bifurcated net 924A. First bifurcated net 924A may be an opening within first limb 908A or second limb 912A of first bifurcated suture 904A. In some embodiments, first bifurcated suture 904A may have two or more bifurcated nets 920A. First bifurcated net 924A may have a length of about 5 mm to about 10 mm. First bifurcated net 924A may have a width of about 5 mm to about 10 mm. In some embodiments, first limb 908B of second bifurcated suture 908B passes through first bifurcated net 924A. Second limb 912B of second bifurcated suture 908B may have second bifurcated net 924B, which may have the same dimensions of first bifurcated net 924A. In some embodiments, first and second bifurcated nets 924A and 924B may have differing dimensions. Second bifurcated suture 904B may have tow or more bifurcated nets 924B. Second limb 912A of first bifurcated suture 904A may pass through second bifurcated net 924B. First limb 908B of second bifurcated suture 904B may pass through first bifurcated net 924A and continue on to pass towards second anchor insert 920B. For instance, first limb 908B of second bifurcated suture 904B may pass in a first direction towards first anchor insert 920A passing through first bifurcated net 920A and then pivot towards a second direction to pass towards second anchor insert 920B. Second limb 912A of first bifurcated suture 904A may pass in a first direction towards second anchor insert 920B through second bifurcated net 924B and may pivot in a second direction towards first anchor insert 920A. In some embodiments, limbs of first bifurcated suture 904A and/or second bifurcated suture 904B may pass through multiple bifurcated nets of each other. Bifurcated nets 920A and 920B may be the same as “openings” described herein.

Second limb 912A of first bifurcated suture 904A may provide a tension on second limb 912B of second bifurcated suture 904B. For instance and without limitation, second limb 912A may pull second limb 912B leftwards, such as towards first anchor insert 920A. Likewise, first limb 908B may provide a tension to first limb 908A towards a right side of first bifurcated suture 904A, such as towards second anchor insert 920B. A weaving or passing of limbs of first and second bifurcated sutures 904A and 904B may form suture net 928. Suture net 928 may be diamond shaped or other shapes, depending on a quantity of bifurcated nets used. As described herein, therefore, the ends of the suture systems may be anchored to bone using bone anchors (e.g. bone anchor inserts such as 920A, 920B,) , or to soft tissue using a suture anchor insert, or suture insert (e.g. 916A, 916B). Thus, elements noted as 102a, 102b, 104a, 104b, herein may be adapted to be coupled to bone or to the soft tissue, depending on the particular need, location, and defect to be addressed in the patient, by swapping bone anchors (or bone anchor inserts) for suture anchors, or suture anchor inserts as described herein.

Referring now to FIG. 11A, a bottom surface of the suture system illustrated in FIG. 10 is shown. As noted above with reference to FIG. 10, first limb 908A and second limb 912 B may have first and second bifurcated nets 920A and 920B, respectively, which may form suture net 928.

Referring now to FIG. 11B, a top surface of the suture system illustrated in FIG. 11 is shown. At a top view with respect to a soft tissue of a patient, the limbs of first and second bifurcated sutures 904A and 904B may be hidden beneath the soft tissue. First suture anchor insert 916A and second suture anchor insert 904B may be visible above a surface of soft tissue 932. First anchor insert 920A and second anchor insert 920B may be visible above a surface of soft tissue 932. First anchor insert 920A and/or second anchor insert 920B may be positioned adjacent to soft tissue 932, such as within about 10 mm, about 9 mm, about 8 mm, about 7 mm, about 6 mm, about 5 mm, about 4 mm, about 3 mm, about 2 mm, about 1 mm, or less than about 1 mm from a surface of soft tissue 932. First and second suture anchor inserts 916A and 916B may be inserted into a surface of soft tissue 932. A tension between suture anchor inserts 916A and 916B and anchor inserts 920A and 920B may secure soft tissue 932 to a surrounding bone location of a patient.

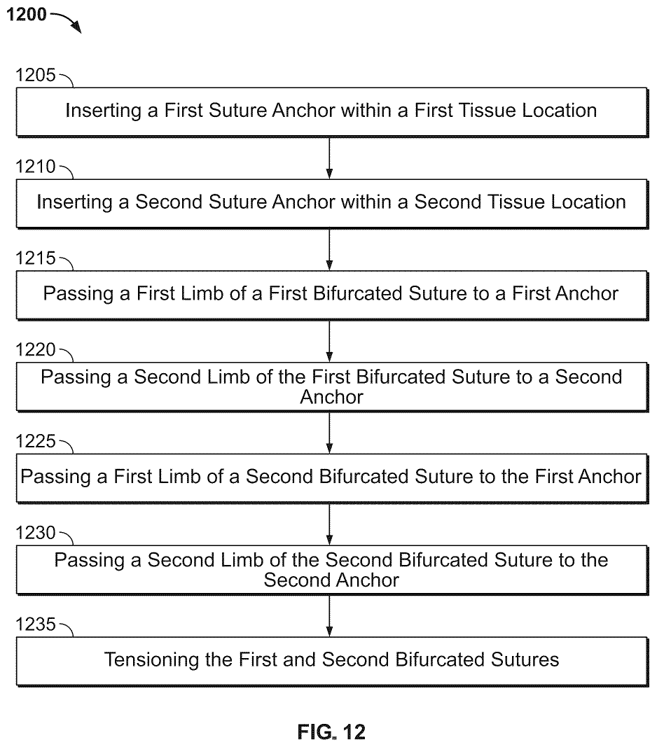

Referring now to FIG. 12, a method of securing two or more bifurcated sutures is presented. At step 1205, method 1200 includes inserting a first suture anchor within a first soft tissue location. A first suture anchor may be inserted into a hole of a first soft tissue location. In some embodiments, a first suture anchor is deployed once inserted into a first soft tissue location. A deployment of a first suture anchor may include transitioning the first suture anchor from a pre-deployment configuration to a deployment configuration. A deployment configuration may include one or more knots or balls of suture bundled close together to form an anchor. A first suture anchor may be deployed via a pulling one a suture tail of the first suture anchor. An anchor formed by knots or bundles of suture of a first suture anchor may have a width greater than a width of a hole of the first soft tissue location, which may secure the first suture anchor to the first soft tissue location. This step may be implemented without limitations as described above with reference to FIGS. 1-11B.

At step 1210, method 1200 includes inserting a second suture anchor within a second soft tissue location. A second suture anchor may be inserted into a hole of a second soft tissue location. A second soft tissue location may be adjacent to a first soft tissue location. A first and second soft tissue location may be part of the same soft tissue. In some embodiments, a second suture anchor is deployed once inserted into a second soft tissue location. A deployment of a second suture anchor may include transitioning the second suture anchor from a pre-deployment configuration to a deployment configuration. A deployment configuration may include one or more knots or balls of suture bundled close together to form an anchor. A second suture anchor may be deployed via a pulling one a suture tail of the second suture anchor. An anchor formed by knots or bundles of suture of a second suture anchor may have a width greater than a width of a hole of the second soft tissue location, which may secure the second suture anchor to the second soft tissue location. This step may be implemented without limitations as described above with reference to FIGS. 9-11B.

At step 1215, method 1200 includes passing a first limb of a first bifurcated suture to a first anchor. A first limb of the first bifurcated suture may be passed at a substantially straight orientation relative to a first suture anchor. A first limb of the first bifurcated suture may be passed towards a first anchor. A first anchor may be inserted into a first bone location and may be positioned across from a first suture anchor with respective to a horizontal axis. This step may be implemented without limitations as described above with reference to FIGS. 9-11B.

At step 1220, method 1200 includes passing a second limb of the first bifurcated suture to a second anchor. A second limb of the first bifurcated suture may be passed towards a second a anchor at an angle. For instance and without limitation, a second limb of the first bifurcated suture may be angled at about 45 degrees, greater than about 45 degrees, or less than about 45 degrees relative to a first suture anchor insert. A second anchor insert may be positioned in a second bone location of a subject. A second anchor insert may be positioned at a same distance to a second suture anchor relative to a distance between a first anchor and a first suture anchor. This step may be implemented without limitations as described above with reference to FIGS. 9-11B.

At step 1225, method 1200 includes passing a first limb of a second bifurcated suture to the first anchor. A first limb of the second bifurcated suture may be passed towards a first anchor at an angle. For instance and without limitation, a first limb of the second bifurcated suture may be angled at about 45 degrees, greater than about 45 degrees, or less than about 45 degrees relative to a second suture anchor insert. In some embodiments, passing the first limb of the second bifurcated suture includes crossing over or under a second limb of the first bifurcated suture. A crossing or weaving of arms of a first and second bifurcated suture may form a suture net. This step may be implemented without limitations as described above with reference to FIGS. 9-11B.

At step 1230, method 1200 includes passing a second limb of the second bifurcated suture to the second anchor. The second limb of the second bifurcated suture may be passed at a substantially straight orientation relative to the second anchor. This step may be implemented without limitations as described above with reference to FIGS. 9-11B.

At step 1235, method 1200 includes tensioning the first and second bifurcate sutures. The first and second bifurcated sutures may be tensioned via a pulling one a respective tensional tail of each of the first and second bifurcated sutures. This step may be implemented without limitations as described above with reference to FIGS. 9-11B.

In some embodiments, method 1200 includes passing a second limb of a first bifurcated suture through a bifurcated net of a first limb of a second bifurcated suture. Method 1200 may include passing a first limb of a second bifurcated suture through a second bifurcated net of a first limb of a first bifurcated suture. In some embodiments, multiple bifurcated nets and weaving thereof between multiple limbs of first and second bifurcated sutures is implemented. For instance, a second limb of a first bifurcated suture may pass through two or more bifurcated nets of a first limb of a second bifurcated suture. Likewise, in some embodiments, a first limb of a second bifurcated suture passes through multiple bifurcated nets of a first limb of a first bifurcated suture.

As used herein, the term “approximately” or “about” means within 15% of a given stated numerical value for a parameter, variable, dimension, and the like.

As will be understood by one of ordinary skill in the art, any of the anchors, inserts, sutures, and delivery devices may be combined with one another or substituted for another and thus any number of combinations may be used. Additionally, various features of an anchor system have been described herein including anchor tip configurations, insert tip configurations, channel configurations, suture passage configurations, suture groove configurations, bone engaging ridges, suture types and numbers, delivery device configurations, driver tip configurations, and device coupler configurations. One of ordinary skill in the art will appreciate that these features may be combined with one another or substituted for another and thus any number of combinations may be used.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. : A suture anchor system comprising:
(a) a suture having a first end, a second end, and a first opened section therebetween, the first opened section defining a first leg, a second leg, and a first opening therebetween; and
(b) a plurality of anchors, each configured to be independently inserted within a respective tissue of a subject about a repair region thereof, the plurality of anchors comprising a first, second, and third anchor;
wherein:
(i) the first anchor and the second anchor are configured to secure a primary segment of the suture to the respective tissue, the primary segment including the first end and the first leg of the first opened section; and
(ii) the second anchor and the third anchor are configured to secure a secondary segment of the suture, the secondary segment extending from the second anchor, passing through the first opening, and through the third anchor to the second end to form a first suture net;
such that tensioning the second end when passed through the third anchor transitions the first opened section from a relaxed configuration to a tensioned configuration, thereby allowing a first compressive force to be applied about the repair region by the formed first suture net including the primary segment, the secondary segment, and the second leg of the first opened section.

2. The suture anchor system of claim 1, wherein tensioning the second end causes the secondary segment to interact with the second leg of the first opened section, thereby enlarging the first opening defined within the first opened section.

3. The suture anchor system of claim 1, wherein the first compressive force applied is adjustable based on the tensioning of the second end, a first distance between the first anchor and the second anchor, a second distance between the second anchor and the third anchor, or any combination thereof.

4. The suture anchor system of claim 1, wherein one or more of the first, second, and third anchors are secured to the respective tissue using an all-suture assembly.

5. The suture anchor system of claim 1, wherein one or more of the first, second, and third anchors are secured to the respective tissue using a knotless suture assembly.

6. The suture anchor system of claim 1, wherein the first anchor and the third anchor are disposed on a lateral row of a humerus, and the second anchor is disposed on a medial row of the humerus.

7. The suture anchor system of claim 1, wherein the suture comprises a second opened section configured to receive the secondary segment therethrough.

8. The suture anchor system of claim 7, wherein the second opened section comprises (i) a third leg disposed along the primary segment, (ii) a fourth leg, and (iii) a second opening therebetween, and wherein the secondary segment extending from the second anchor, passing through the first opening, second opening, and through the third anchor to the second end such that tensioning the second end when passed through the third anchor allows for the first compressive force to be applied about the repair region by the primary segment, the secondary segment, the second leg, and the fourth leg.

9. The suture anchor system of claim 1, wherein the suture comprises three or more opened sections.

10. The suture anchor system of claim 1, wherein at least one opened section is a bifurcation of the suture.

11. The suture anchor system of claim 1, wherein at least one opened section comprises a first separate suture strand affixed to the suture at a proximal and distal location, thereby forming a respective opening between the suture and the first separate strand, the first separate strand therefore acting as a respective leg for said at least one opened section.

12. The suture anchor system of claim 11, wherein the first separate strand is affixed to the suture via tying, braiding, melting, an adhesive, or any combination thereof.

13. The suture anchor system of claim 1, wherein the second leg is longer than the first leg.

14. The suture anchor system of claim 1, further comprising a second suture having a third end, a fourth end, and a third opened section therebetween, the third opened section defining a fifth leg, a sixth leg, and a third opening therebetween wherein:
(i) the third anchor and the second anchor are configured to secure a primary segment of the second suture to the respective tissue, the primary segment of the second suture including the third end and the fifth leg of the third opened section; and
(ii) the second anchor and the first anchor are configured to secure a secondary segment of the second suture, the secondary segment of the second suture extending from the second anchor, through the third opened section, and through the first anchor to the fourth end to form a second suture net;
such that tensioning the fourth end when passed through the first anchor transitions the third opened section from a relaxed configuration to a tensioned configuration, thereby allowing a second compressive force to be applied about the repair region by the formed second suture net including the primary segment of the second suture, the secondary segment of the second suture, and the sixth leg of the third opened section.

15. The suture anchor system of claim 14, wherein the secondary segment of the second suture is configured to pass through one or both of the first and second opened sections.

16. The suture anchor system of claim 14, wherein tensioning the fourth end causes the secondary segment of the second suture to interact with the sixth leg, thereby enlarging the third opening defined within the third opened section.

17. The suture anchor system of claim 14, wherein the second compressive force applied is adjustable based on the tensioning of the fourth end, a first distance between the first anchor and the second anchor, a second distance between the second anchor and the third anchor, or any combination thereof.

18. The suture anchor system of claim 14, wherein one or more of the third, second, and first anchors are secured to the respective tissue using an all-suture assembly.

19. The suture anchor system of claim 14, wherein one or more of the first, second, and third anchors are secured to the respective tissue using a knotless suture assembly.

20. The suture anchor system of claim 14, wherein the first anchor and the third anchor are disposed on a lateral row of a humerus, and the second anchor is disposed on a medial row of the humerus.

21. The suture anchor system of claim 14, wherein the second suture comprises a fourth opened section configured to receive the secondary segment of the second suture therethrough.

22. The suture anchor system of claim 21, wherein the fourth opened section comprises (i) a seventh leg disposed along the primary segment of the second suture, and (ii) an eighth leg, and wherein the secondary segment of the second suture extending from the second anchor, through the third opened section, the fourth opened section, and through the first anchor to the fourth end such that tensioning the fourth end when passed through the first anchor allows for the second compressive force to be applied about the repair region by the primary segment of the second suture, the secondary segment of the second suture, the sixth leg, and the eighth leg.

23. The suture anchor system of claim 14, wherein the second suture comprises three or more opened sections.

24. The suture anchor system of claim 14, wherein at least one opened section is a bifurcation of the second suture.

25. The suture anchor system of claim 14, wherein at least one opened section comprises a second separate suture strand affixed to the second suture at a proximal and distal location thereof, thereby forming a respective opening between the second suture and the second separate strand, the second separate strand therefore acting as a respective leg for said at least one opened section.

26. The suture anchor system of claim 25, wherein the second separate suture strand is affixed to the second suture via tying, braiding, an adhesive, or any combination thereof.

27. The suture anchor system of claim 1, further comprising a second suture having a third end, a fourth end, and a third opened section therebetween, the third opened section defining a fifth leg, a sixth leg, and a third opening therebetween wherein:
(i) the third anchor and a fourth anchor are configured to secure a primary segment of the second suture to the respective tissue, the primary segment of the second suture including the third end and the fifth leg of the third opened section; and
(ii) the fourth anchor and the first anchor are configured to secure a secondary segment of the second suture, the secondary segment of the second suture extending from the fourth anchor, through the third opened section, and through the first anchor to the fourth end to form a third suture net;
such that tensioning the fourth end when passed through the first anchor transitions the third opened section from a relaxed configuration to a tensioned configuration, thereby allowing a second compressive force to be applied about the repair region by the formed third suture net including the primary segment of the second suture, the secondary segment of the second suture, and the sixth leg of the third opened section.

28. The suture anchor system of claim 27, wherein tensioning the fourth end causes the secondary segment of the second suture to interact with the sixth leg, thereby enlarging the third opening defined within the third opened section.

29. The suture anchor system of claim 27, wherein the second compressive force applied is adjustable based on the tensioning of the fourth end, a first distance between the first anchor and the fourth anchor, a second distance between the fourth anchor and the third anchor, or any combination thereof.

30. The suture anchor system of claim 27, wherein one or more of the third, fourth and first anchors are secured to the respective tissue using an all-suture assembly.

31. The suture anchor system of claim 27, wherein one or more of the first, fourth, and third anchors are secured to the respective tissue using a knotless suture assembly.

32. The suture anchor system of claim 27, wherein the first anchor and the third anchor are disposed on a lateral row of a humerus, and the fourth anchor is disposed on a medial row of the humerus.

33. The suture anchor system of claim 27, wherein the second suture comprises a fourth opened section configured to receive the secondary segment of the second suture therethrough.

34. The suture anchor system of claim 33, wherein the fourth opened section comprises (i) a seventh leg disposed along the primary segment of the second suture, and (ii) an eighth leg, such that tensioning the fourth end when passed through the first anchor allows for the second compressive force to be applied about the repair region by the primary segment of the second suture, the secondary segment of the second suture, the sixth leg, and the eighth leg.

35. The suture anchor system of claim 27, wherein the second suture comprises three or more opened sections.

36. The suture anchor system of claim 27, wherein at least one opened section is a bifurcation of the second suture.

37. The suture anchor system of claim 27, wherein at least one opened section comprises a second separate suture strand affixed to the second suture at a proximal and distal location thereof, thereby forming a respective opening between the second suture and the second separate strand, the second separate strand therefore acting as a respective leg for said at least one opened section.

38. The suture anchor system of claim 37, wherein the second separate suture strand is affixed to the second suture via tying, braiding, an adhesive, or any combination thereof.

39-49. (canceled)

50. A method of securing one or more sutures, the method comprising:
a) inserting a second anchor within a bone of a subject;
b) passing a suture through the second anchor, the suture having a first end and second end extending from the second anchor;
c) passing the first and second ends of the suture through a soft tissue to be secured to the bone, such that the suture passes from a bone facing portion of the soft tissue and through an aperture to an opposite facing portion of the soft tissue;
d) securing the first end of the suture through a first anchor that is then inserted into the bone, wherein the suture from the first end to the aperture comprises an opened section having a first leg, a second leg, and an opening therebetween, the suture defining a primary segment from the first end to the aperture and comprising the first leg;
e) passing at least a portion of a secondary segment of the suture through the opening of the opened section and then through a third anchor that is then inserted into the bone to form a suture net, the secondary segment comprising the suture from the second end to the aperture;
f) tensioning the secondary segment so as to transition the opened section from a relaxed configuration to a tensioned configuration via interaction between the secondary segment and the second leg, such that a compressive force is applied about the tissue on the bone by the formed suture net including the primary segment, the secondary segment, and the second leg; and
g) securing the second end to the bone via one or more of the first, second, and third anchors.

51. The method of claim 50, further comprising forming one or more additional suture nets using any combination of the first, the second, the third, and/or one or more additional anchors.

52-78. (canceled)
